(19) United States
(12) Patent Application Publication
(10) Pub. No.: US 2024/0110167 A1
(43) Pub. Date: Apr. 4, 2024

(54) ENZYMES WITH RUVC DOMAINS

(71) Applicant: Metagenomi, Inc., Emeryville, CA (US)

(72) Inventors: Brian C. THOMAS, Berkeley, CA (US); Christopher BROWN, Albany, CA (US); Daniela S.A. GOLTSMAN, Oakland, CA (US); Cristina BUTTERFIELD, Oakland, CA (US); Lisa ALEXANDER, Albany, CA (US); Cindy CASTELLE, San Francisco, CA (US)

(21) Appl. No.: 18/488,520

(22) Filed: Oct. 17, 2023

Related U.S. Application Data
(63) Continuation of application No. PCT/US2022/027124, filed on Apr. 29, 2022.
(60) Provisional application No. 63/182,438, filed on Apr. 30, 2021.

(51) Int. Cl. C12N 9/22; C12N 15/10; C12N 15/113

(52) U.S. Cl. CPC C12N 9/22; C12N 15/102; C12N 15/113

(57) ABSTRACT

The present disclosure provides for endonuclease enzymes having distinguishing domain features, as well as methods of using such enzymes or variants thereof.

### CROSS-REFERENCE

This application is a continuation of International Application No. PCT/US2022/027124, filed Apr. 29, 2022, which claims the benefit of U.S. Provisional Application No. 63/182,438, filed on Apr. 30, 2021, each of which is incorporated by reference herein in its entirety.

### BACKGROUND

Cas enzymes along with their associated Clustered Regularly Interspaced Short Palindromic Repeats (CRISPR) guide ribonucleic acids (RNAs) appear to be a pervasive (˜45% of bacteria, ˜84% of archaea) component of prokaryotic immune systems, serving to protect such microorganisms against non-self nucleic acids, such as infectious viruses and plasmids by CRISPR-RNA guided nucleic acid cleavage. While the deoxyribonucleic acid (DNA) elements encoding CRISPR RNA elements may be relatively conserved in structure and length, their CRISPR-associated (Cas) proteins are highly diverse, containing a wide variety of nucleic acid-interacting domains. While CRISPR DNA elements have been observed as early as 1987, the programmable endonuclease cleavage ability of CRISPR/Cas complexes has only been recognized relatively recently, leading to the use of recombinant CRISPR/Cas systems in diverse DNA manipulation and gene editing applications.

### SEQUENCE LISTING

The instant application contains a Sequence Listing which has been submitted electronically in XML format and is hereby incorporated by reference in its entirety. Said XML copy, created on Oct. 12, 2023, is named 55921_722_601_Sequence_Listing_Final.xml and is 3,588,096 bytes in size.

### SUMMARY

In some aspects, the present disclosure provides for an engineered nuclease system comprising:(a) an endonuclease configured to bind to a protospacer adjacent motif (PAM) sequence comprising SEQ ID NOs: 550-567, wherein said endonuclease is a class 2, type II Cas endonuclease; and (b) an engineered guide ribonucleic acid structure configured to form a complex with said endonuclease comprising: (i) a guide ribonucleic acid sequence configured to hybridize to a target deoxyribonucleic acid sequence; and (ii) a tracr ribonucleic acid sequence configured to bind to said endonuclease. In some embodiments, said endonuclease is derived from an uncultivated microorganism. In some embodiments, said endonuclease has not been engineered to bind to a different PAM sequence. In some embodiments, said endonuclease is not a Cas9 endonuclease, a Cas14 endonuclease, a Cas12a endonuclease, a Cas12b endonuclease, a Cas 12c endonuclease, a Cas12d endonuclease, a Cas12e endonuclease, a Cas13a endonuclease, a Cas13b endonuclease, a Cas13c endonuclease, or a Cas 13d endonuclease. In some embodiments, said endonuclease has less than 80% identity to a Cas9 endonuclease. In some embodiments, said endonuclease further comprises an HNH domain In some embodiments, said engineered guide ribonucleic acid structure comprises at least two ribonucleic acid polynucleotides. In some embodiments, said engineered guide ribonucleic acid structure comprises one ribonucleic acid polynucleotide comprising said guide ribonucleic acid sequence and said tracr ribonucleic acid sequence. In some embodiments, said guide ribonucleic acid sequence is complementary to a prokaryotic, bacterial, archaeal, eukaryotic, fungal, plant, mammalian, or human genomic sequence. In some embodiments, said guide ribonucleic acid sequence is 15-24 nucleotides in length. In some embodiments, said endonuclease comprises one or more nuclear localization sequences (NLSs) proximal to an N- or C-terminus of said endonuclease. In some embodiments, said NLS comprises a sequence selected from SEQ ID NOs: 586-601. In some embodiments, the engineered nuclease system further comprises a single- or double-stranded DNA repair template comprising from 5′ to 3′: a first homology arm comprising a sequence of at least 20 nucleotides 5′ to said target deoxyribonucleic acid sequence, a synthetic DNA sequence of at least 10 nucleotides, and a second homology arm comprising a sequence of at least 20 nucleotides 3′ to said target sequence. In some embodiments, said first or second homology arm comprises a sequence of at least 40, 80, 120, 150, 200, 300, 500, or 1,000 nucleotides. In some embodiments, said system further comprises a source of Mg²⁺. In some embodiments, said endonuclease and said tracr ribonucleic acid sequence are derived from distinct bacterial species within a same phylum. In some embodiments, said endonuclease comprises SEQ ID NOs: 1-549 or 602-1276, or a variant thereof having at least 55% identity thereto.

In one aspect, the present disclosure provides for an engineered nuclease system comprising: (a) an endonuclease configured to have selectivity for a protospacer adjacent motif (PAM) sequence comprising any one of SEQ ID NOs: 550-567, wherein the endonuclease is a class 2, type II Cas endonuclease. In some embodiments, the system further comprises (b) an engineered guide nucleic acid structure configured to form a complex with the endonuclease comprising: (i) a targeting nucleic acid sequence configured to hybridize to a target deoxyribonucleic acid sequence; and (ii) a tracr ribonucleic acid sequence configured to bind to the endonuclease. In some embodiments, the endonuclease is derived from an uncultivated microorganism. In some embodiments, the endonuclease has not been engineered to bind to a different PAM sequence than a native PAM sequence of the endonuclease. In some embodiments, the endonuclease is not a Cas9 endonuclease, a Cas14 endonuclease, a Cas12a endonuclease, a Cas12b endonuclease, a Cas 12c endonuclease, a Cas12d endonuclease, a Cas12e endonuclease, a Cas13a endonuclease, a Cas13b endonuclease, a Cas13c endonuclease, or a Cas 13d endonuclease. In some embodiments, the endonuclease has less than 80% identity to a Cas9 endonuclease. In some embodiments, the endonuclease further comprises a PI domain comprising a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1277-1641 or 1683, or a variant thereof. In some embodiments, the endonuclease further comprises a PI domain having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to a PI domain of any one of SEQ ID NOs: 1-549 or 602-1276. In some embodiments, the endonuclease further comprises a PI domain comprising a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1, 217, 258, 259, 284, 296, 297, 306, 357, 403, 404, 405, 463, 464, 465, 356, or 484, or a variant thereof. In some embodiments, the endonuclease further comprises a PI domain comprising a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 259, 296, or 484, or a variant thereof. In some embodiments, the endonuclease further comprises a RuvC domain In some embodiments, the RuvC domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to a RuvC domain of any one of SEQ ID NOs: 1, 217, 258, 259, 284, 296, 297, 306, 357, 403, 404, 405, 463, 464, 465, 356, or 484, or a variant thereof. In some embodiments, the RuvC domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to a RuvC domain of any one of SEQ ID NOs: 259, 296, or 484, or a variant thereof. In some embodiments, the endonuclease further comprises an HNH domain In some embodiments, the HNH domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to an HNH domain of any one of SEQ ID NOs: 1, 217, 258, 259, 284, 296, 297, 306, 357, 403, 404, 405, 463, 464, 465, 356, or 484, or a variant thereof. In some embodiments, the HNH domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to an HNH domain of any one of SEQ ID NOs: 259, 296, or 484, or a variant thereof. In some embodiments, the endonuclease is configured to have selectivity for a PAM sequence comprising any one of SEQ ID NOs: 553, 555, or 566, or a variant thereof. In some embodiments, the engineered guide nucleic acid structure comprises at least two ribonucleic acid polynucleotides. In some embodiments, the engineered guide nucleic acid structure comprises one ribonucleic acid polynucleotide comprising the guide ribonucleic acid sequence and the tracr ribonucleic acid sequence. In some embodiments, the engineered guide nucleic acid structure comprises a tracr sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1645-1662 or wherein the engineered guide nucleic acid structure comprises a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to non-degenerate nucleotides of any one of SEQ ID NOs: 568-585 or 1643-1644. In some embodiments, the engineered guide nucleic acid structure comprises a tracr sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1648, 1650, or 1661 or wherein the engineered guide nucleic acid structure comprises a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to non-degenerate nucleotides of any one of SEQ ID NOs: 571, 573, or 584. In some embodiments, the targeting nucleic acid sequence is complementary to a prokaryotic, bacterial, archaeal, eukaryotic, fungal, plant, mammalian, or human genomic sequence. In some embodiments, the targeting nucleic acid sequence is 15-24 nucleotides in length. In some embodiments, the endonuclease comprises one or more nuclear localization sequences (NLSs) proximal to an N- or C-terminus of the endonuclease. the NLS comprises a sequence comprising any one of SEQ ID NOs: 586-601, or a variant thereof. In some embodiments, the system further comprises a single- or double-stranded DNA repair template comprising from 5′ to 3′: a first homology arm comprising a sequence of at least 20 nucleotides 5′ to the target deoxyribonucleic acid sequence, a synthetic DNA sequence of at least 10 nucleotides, and a second homology arm comprising a sequence of at least 20 nucleotides 3′ to the target sequence. In some embodiments, the first or second homology arm comprises a sequence of at least 40, 80, 120, 150, 200, 300, 500, or 1,000 nucleotides. In some embodiments, the system further comprises a source of Mg²⁺. In some embodiments, the endonuclease and the tracr ribonucleic acid sequence are derived from distinct bacterial species within a same phylum. In some embodiments, the endonuclease comprises any one of SEQ ID NOs: 1, 217, 258, 259, 284, 296, 297, 306, 357, 403, 404, 405, 463, 464, 465, 356, or 484:or a variant thereof having at least 55% identity thereto. In some embodiments, the sequence identity is determined by a BLASTP, CLUSTALW, MUSCLE, MAFFT algorithm, or a CLUSTALW algorithm with the Smith-Waterman homology search algorithm parameters. In some embodiments, the sequence identity is determined by the BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment. In some embodiments, the PAM sequence is 3′ to the target deoxyribonucleic acid sequence

In some aspects, the present disclosure provides for a nucleic acid comprising an engineered nucleic acid sequence optimized for expression in an organism, wherein the nucleic acid encodes a class 2, type II Cas endonuclease configured to be selective for a protospacer adjacent motif (PAM) comprising any one of SEQ ID NOs: 550-567. In some embodiments, the endonuclease further comprises a PI domain comprising a sequence having at least at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1277-1641 or 1683, or a variant thereof, or wherein the endonuclease further comprises a PI domain having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to a PI domain of any one of SEQ ID NOs: 1-549 or 602-1276. In some embodiments, the endonuclease further comprises a PI domain comprising a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1, 217, 258, 259, 284, 296, 297, 306, 357, 403, 404, 405, 463, 464, 465, 356, or 484, or a variant thereof. In some embodiments, the endonuclease further comprises a PI domain comprising a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 259, 296, or 484, or a variant thereof. In some embodiments, the endonuclease further comprises a RuvC domain In some embodiments, the RuvC domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to a RuvC domain of any one of SEQ ID NOs: 1, 217, 258, 259, 284, 296, 297, 306, 357, 403, 404, 405, 463, 464, 465, 356, or 484, or a variant thereof. In some embodiments, the RuvC domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to a RuvC domain of any one of SEQ ID NOs: 259, 296, or 484, or a variant thereof. In some embodiments, the endonuclease further comprises an HNH domain. In some embodiments, the HNH domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to an HNH domain of any one of SEQ ID NOs: 1, 217, 258, 259, 284, 296, 297, 306, 357, 403, 404, 405, 463, 464, 465, 356, or 484, or a variant thereof. In some embodiments, the HNH domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to an HNH domain of any one of SEQ ID NOs: 259, 296, or 484, or a variant thereof. In some embodiments, the endonuclease comprises any one of SEQ ID NOs: 1, 217, 258, 259, 284, 296, 297, 306, 357, 403, 404, 405, 463, 464, 465, 356, or 484, or a variant thereof having at least 55%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity thereto. In some embodiments, the organism is a bacterial, archaeal, eukaryotic, fungal, plant, mammalian, or human organism,

In some aspects, the present disclosure provides for an engineered nuclease system comprising: (a) an endonuclease having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1-549, 602-1276, or a variant thereof; and (b) an engineered guide nucleic acid structure, wherein the engineered guide RNA is configured to form a complex with the endonuclease and the engineered guide RNA comprises a targeting nucleic acid sequence configured to hybridize to a target nucleic acid sequence. In some embodiments, the endonuclease comprises any one of SEQ ID NOs: 1, 217, 258, 259, 284, 296, 297, 306, 357, 403, 404, 405, 463, 464, 465, 356, or 484, or a variant thereof having at least 55% identity, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity thereto. In some embodiments, the endonuclease further comprises a RuvC domain In some embodiments, the RuvC domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to a RuvC domain of any one of SEQ ID NOs: 1, 217, 258, 259, 284, 296, 297, 306, 357, 403, 404, 405, 463, 464, 465, 356, or 484, or a variant thereof. In some embodiments, the RuvC domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to a RuvC domain of any one of SEQ ID NOs: 259, 296, or 484, or a variant thereof In some embodiments, the endonuclease further comprises an HNH domain In some embodiments, the HNH domain at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to an HNH domain of any one of SEQ ID NOs: 1, 217, 258, 259, 284, 296, 297, 306, 357, 403, 404, 405, 463, 464, 465, 356, or 484, or a variant thereof. In some embodiments, the HNH domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to an HNH domain of any one of SEQ ID NOs: 259, 296, or 484, or a variant thereof. In some embodiments, the engineered guide nucleic acid structure comprises a tracr sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1645-1662 or wherein the engineered guide nucleic acid structure comprises a sequence having at least 80% identity to non-degenerate nucleotides of any one of SEQ ID NOs: 568-585 or 1643-1644. In some embodiments, the engineered guide nucleic acid structure comprises a tracr sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1648, 1650, or 1661 or wherein the engineered guide nucleic acid structure comprises a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to non-degenerate nucleotides of any one of SEQ ID NOs: 571, 573, or 584. In some embodiments, the engineered guide nucleic acid structure is configured to form a complex with the endonuclease and comprises (i) a targeting nucleic acid sequence configured to hybridize to a target deoxyribonucleic acid sequence; and (ii) a tracr ribonucleic acid sequence configured to bind to the endonuclease. In some embodiments, the targeting nucleic acid sequence is complementary to a bacterial, archaeal, eukaryotic, fungal, plant, mammalian, or human genomic sequence. In some embodiments, the targeting nucleic acid sequence is 15-24 nucleotides in length.

In some aspects, the present disclosure provides for an engineered nuclease system comprising: (a) an engineered guide nucleic acid structure comprising: (i) a tracr sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1645-1662; or (ii) a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to non- degenerate nucleotides of any one of SEQ ID NOs: 568-585 or 1643-1644, and (b) a class 2, type II Cas endonuclease configured to bind to the engineered guide nucleic acid structure. In some embodiments, the engineered guide nucleic acid structure comprises a tracr sequence having at least 80% identity to any one of SEQ ID NOs: 1648, 1650, or 1661 or wherein the engineered guide nucleic acid structure comprises a sequence having at least 80% identity to non-degenerate nucleotides of any one of SEQ ID NOs: 571, 573, or 584. In some embodiments, the engineered guide nucleic acid structure is configured to form a complex with the endonuclease and comprises (i) a targeting nucleic acid sequence configured to hybridize to a target deoxyribonucleic acid sequence; and (ii) a tracr ribonucleic acid sequence configured to bind to the endonuclease. In some embodiments, the targeting nucleic acid sequence is complementary to a bacterial, archaeal, eukaryotic, fungal, plant, mammalian, or human genomic sequence. In some embodiments, the targeting nucleic acid sequence is 15-24 nucleotides in length. In some embodiments, the endonuclease comprises a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1-549, 602-1276, or a variant thereof. In some embodiments, the endonuclease comprises a sequence according to any one of SEQ ID NOs: 1, 217, 258, 259, 284, 296, 297, 306, 357, 403, 404, 405, 463, 464, 465, 356, or 484, or a variant thereof having at least 55%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity thereto.

In some aspects, the present disclosure provides for an engineered guide nucleic acid structure comprising: (a) a targeting nucleic acid sequence comprising a nucleotide sequence that is complementary to a target sequence in a target DNA molecule; and (b) a protein-binding segment comprising two complementary stretches of nucleotides that hybridize to form a double-stranded RNA (dsRNA) duplex, one of which comprising a tracr sequence, wherein the two complementary stretches of nucleotides are covalently linked to one another with intervening nucleotides, and wherein the engineered guide ribonucleic acid polynucleotide is capable of forming a complex with an endonuclease having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1-549, 602-1276, or a variant thereof, and targeting the complex to the target sequence of the target DNA molecule. In some embodiments, the endonuclease comprises any one of SEQ ID NOs: 1, 217, 258, 259, 284, 296, 297, 306, 357, 403, 404, 405, 463, 464, 465, 356, or 484, or a variant thereof having at least 55%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity thereto. In some embodiments, the targeting nucleic acid sequence is complementary to a bacterial, archaeal, eukaryotic, fungal, plant, mammalian, or human genomic sequence. In some embodiments, the targeting nucleic acid sequence is 15-24 nucleotides in length. In some embodiments, the engineered guide nucleic acid structure comprises a tracr sequence having at least 80% identity to any one of SEQ ID NOs: 1645-1662 or wherein the engineered guide nucleic acid structure comprises a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to non-degenerate nucleotides of any one of SEQ ID NOs: 568-585 or 1643-1644. In some embodiments, the engineered guide nucleic acid structure comprises a tracr sequence having at least 80% identity to any one of SEQ ID NOs: 1648, 1650, or 1661 or wherein the engineered guide nucleic acid structure comprises a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to non-degenerate nucleotides of any one of SEQ ID NOs: 571, 573, or 584.

In some aspects, the present disclosure provides for an engineered vector comprising any of the nucleic acids described herein. In some embodiments, the vector is a plasmid, a minicircle, a CELiD, an adeno-associated virus (AAV) derived virion, a lentivirus, or an adenovirus

In some aspects, the present disclosure provides for a cell comprising any of the vectors described herein or any of the nucleic acids described herein. In some embodiments, the cell is a bacterial, archaeal, eukaryotic, fungal, plant, mammalian, or human cell.

In some aspects, the present disclosure provides for a method of manufacturing an endonuclease, comprising cultivating any of the cells described herein.

In some aspects, the present disclosure provides for a method for binding, cleaving, marking, or modifying a double-stranded deoxyribonucleic acid polynucleotide, comprising: contacting the double-stranded deoxyribonucleic acid polynucleotide with a class 2, type II Cas endonuclease in complex with an engineered guide nucleic acid structure configured to bind to the endonuclease and the double-stranded deoxyribonucleic acid polynucleotide; wherein the double-stranded deoxyribonucleic acid polynucleotide comprises a protospacer adjacent motif (PAM); and wherein the PAM comprises a sequence according to any one of SEQ ID NOs: 550-567. In some embodiments, the endonuclease further comprises a PI domain comprising a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1277-1641 or 1683, or a variant thereof, or wherein the endonuclease further comprises a PI domain having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to a PI domain of any one of SEQ ID NOs: 1-549 or 602-1276. In some embodiments, the endonuclease further comprises a PI domain comprising a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1, 217, 258, 259, 284, 296, 297, 306, 357, 403, 404, 405, 463, 464, 465, 356, or 484, or a variant thereof. In some embodiments, the endonuclease further comprises a PI domain comprising a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 259, 296, or 484, or a variant thereof. In some embodiments, the endonuclease further comprises a RuvC domain. In some embodiments, the RuvC domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to a RuvC domain of any one of SEQ ID NOs: 1, 217, 258, 259, 284, 296, 297, 306, 357, 403, 404, 405, 463, 464, 465, 356, or 484, or a variant thereof. In some embodiments, the RuvC domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to a RuvC domain of any one of SEQ ID NOs: 259, 296, or 484, or a variant thereof. In some embodiments, the endonuclease further comprises an HNH domain. In some embodiments, the HNH domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to an HNH domain of any one of SEQ ID NOs: 1, 217, 258, 259, 284, 296, 297, 306, 357, 403, 404, 405, 463, 464, 465, 356, or 484, or a variant thereof. In some embodiments, the HNH domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to an HNH domain of any one of SEQ ID NOs: 259, 296, or 484, or a variant thereof. In some embodiments, the endonuclease comprises any one of SEQ ID NOs: 1, 217, 258, 259, 284, 296, 297, 306, 357, 403, 404, 405, 463, 464, 465, 356, or 484, or a variant thereof having at least 55%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity thereto. In some embodiments, the engineered guide nucleic acid structure comprises a tracr sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1645-1662 or wherein the engineered guide nucleic acid structure comprises a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to non- degenerate nucleotides of any one of SEQ ID NOs: 568-585 or 1643-1644. In some embodiments, the engineered guide nucleic acid structure comprises a tracr sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1648, 1650, or 1661 or wherein the engineered guide nucleic acid structure comprises a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to non-degenerate nucleotides of any one of SEQ ID NOs: 571, 573, or 584. In some embodiments, the engineered guide nucleic acid structure is configured to form a complex with the endonuclease and comprises (i) a targeting nucleic acid sequence configured to hybridize to a target deoxyribonucleic acid sequence; and (ii) a tracr ribonucleic acid sequence configured to bind to the endonuclease. In some embodiments, the targeting nucleic acid sequence is complementary to a bacterial, archaeal, eukaryotic, fungal, plant, mammalian, or human genomic sequence. In some embodiments, the targeting nucleic acid sequence is 15-24 nucleotides in length.

In some aspects, the present disclosure provides for a method of editing an AAVS1 locus in a cell, comprising contacting to the cell (a) an RNA-guided endonuclease; and (b) an engineered guide nucleic acid structure, wherein the engineered guide nucleic acid structure is configured to form a complex with the endonuclease and the engineered guide nucleic acid structure comprises a spacer sequence configured to hybridize to a region of the AAVS1 locus, wherein the engineered guide nucleic acid structure comprises a targeting sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to at least 18 consecutive nucleotides of any one of SEQ ID NOs: 1665-1666 or a reverse complement thereof. In some embodiments, the engineered guide nucleic acid structure has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NO: 1663 or 1664. In some embodiments, the engineered guide nucleic acid structure is MG71-2-AAVS1-sgRNA-C3 or MG71-2-AAVS1-sgRNA-E2.

In some aspects, the present disclosure provides for a method of editing a TRAC locus in a cell, comprising contacting to the cell (a) an RNA-guided endonuclease; and (b) an engineered guide nucleic acid structure, wherein the engineered guide nucleic acid structure is configured to form a complex with the endonuclease and the engineered guide nucleic acid structure comprises a spacer sequence configured to hybridize to a region of the TRAC locus, wherein the engineered guide nucleic acid structure comprises a targeting sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to at least 18 consecutive nucleotides of any one of SEQ ID NOs: 1668 or 1676-1682, or a reverse complement thereof. In some embodiments, the engineered guide nucleic acid structure has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1667 or 1669-1675. In some embodiments, the engineered guide nucleic acid structure is MG73-1-TRAC-sgRNA-G3, MG89-2-TRAC-sgRNA-F1, MG89-2-TRAC-sgRNA-G5, MG89-2-TRAC-sgRNA-E5, MG89-2-TRAC-sgRNA-F5, MG89-2-TRAC-sgRNA-G1, MG89-2-TRAC-sgRNA-E1, MG89-2-TRAC-sgRNA-B1. In some embodiments, the engineered guide nucleic acid structure is configured to form a complex with the endonuclease and comprises (i) a targeting nucleic acid sequence configured to hybridize to a target deoxyribonucleic acid sequence; and (ii) a tracr ribonucleic acid sequence configured to bind to the endonuclease. In some embodiments, the targeting nucleic acid sequence is 15-24 nucleotides in length. In some embodiments, the engineered guide nucleic acid structure comprises a tracr sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1645-1662 or wherein the engineered guide nucleic acid structure comprises a sequence having at least 80% identity to non- degenerate nucleotides of any one of SEQ ID NOs: 568-585 or 1643-1644. In some embodiments, the engineered guide nucleic acid structure comprises a tracr sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1648, 1650, or 1661 or wherein the engineered guide nucleic acid structure comprises a sequence having at least 80% identity to non-degenerate nucleotides of any one of SEQ ID NOs: 571, 573, or 584. In some embodiments, the RNA guided endonuclease is a class 2, type II Cas endonuclease. In some embodiments, the endonuclease is configured to have selectivity for a PAM sequence comprising any one of SEQ ID NOs: 550-567. In some embodiments, the endonuclease further comprises a PI domain comprising a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1277-1641 or 1683, or a variant thereof, or wherein the endonuclease further comprises a PI domain having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to a PI domain of any one of SEQ ID NOs: 1-549 or 602-1276. In some embodiments, the endonuclease further comprises a PI domain comprising a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1, 217, 258, 259, 284, 296, 297, 306, 357, 403, 404, 405, 463, 464, 465, 356, or 484, or a variant thereof. In some embodiments, the endonuclease further comprises a PI domain comprising a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 259, 296, or 484, or a variant thereof. In some embodiments, the endonuclease comprises any one of SEQ ID NOs: 1, 217, 258, 259, 284, 296, 297, 306, 357, 403, 404, 405, 463, 464, 465, 356, or 484, or a variant thereof having at least 55% identity, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity thereto.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 depicts typical organizations of CRISPR/Cas loci of different classes and types.

FIG. 2 depicts the architecture of a natural Class II/Type II crRNA/tracrRNA pair, compared to a hybrid sgRNA wherein both are joined.

FIG. 3A, FIG. 3B, FIG. 3C, FIG. 3D, and FIG. 3E depict seqLogo representations of PAM sequences derived via NGS as described herein (e.g., as described in Example 3).

FIG. 4 depicts the gene editing outcomes at the DNA level for TRAC and AAVS1 in K562 cells in Example 7.

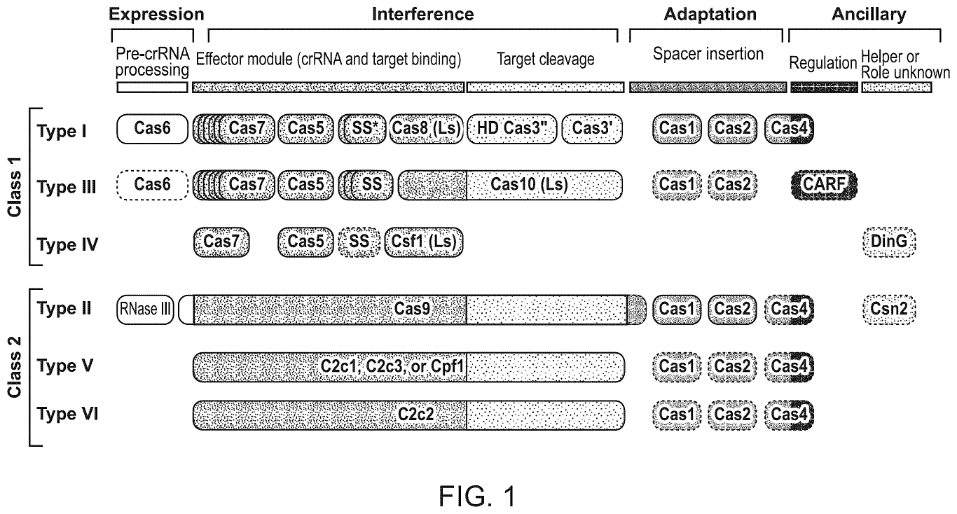

### BRIEF DESCRIPTION OF THE SEQUENCE LISTING

The Sequence Listing filed herewith provides exemplary polynucleotide and polypeptide sequences for use in methods, compositions, and systems according to the disclosure. Below are exemplary descriptions of sequences therein.

### MG44

SEQ ID NOs: 1-216 and 602-938 show the full-length peptide sequences of MG44 nucleases.

SEQ ID NO: 550 shows a PAM sequence compatible with an MG44 nuclease.

SEQ ID NO: 568 shows a nucleotide sequences of sgRNA engineered to function with an MG44 nuclease, where Ns denote nucleotides of a targeting sequence.

SEQ ID NOs: 1277-1419 show the peptide sequences of PAM-interacting domains of MG44 nucleases.

SEQ ID NO: 1645 shows the nucleotide sequence of an MG44 tracrRNA derived from the same loci as MG44 nucleases above.

### MG46

SEQ ID NOs: 217-257 and 939-1104 show the full-length peptide sequences of MG46 nucleases.

SEQ ID NO: 551 shows a PAM sequence compatible with an MG46 nuclease.

SEQ ID NO: 569 shows a nucleotide sequences of sgRNA engineered to function with an MG46 nuclease, where Ns denote nucleotides of a targeting sequence.

SEQ ID NOs: 1420-1497 show the peptide sequences of PAM-interacting domains of MG46 nucleases.

SEQ ID NO: 1646 shows the nucleotide sequence of an MG46 tracrRNA derived from the same loci as MG46 nucleases above.

### MG71

SEQ ID NOs: 258-283 and 1105 show the full-length peptide sequences of MG71 nucleases.

SEQ ID NOs: 552-553 shows PAM sequences compatible with MG71 nucleases.

SEQ ID NOs: 570-571 show nucleotide sequences of sgRNA engineered to function with an MG71 nucleases, where Ns denote nucleotides of a targeting sequence.

SEQ ID NOs: 1498-1499 show the peptide sequences of PAM-interacting domains of MG71 nucleases.

SEQ ID NOs: 1643-1644 show nucleotide sequences of sgRNAs engineered to function with an MG71 nuclease.

SEQ ID NOs: 1647-1648 show nucleotide sequences of MG71 tracrRNAs derived from the same loci as MG71 nucleases above.

### MG72

SEQ ID NOs: 284-295 and 1106-1115 show the full-length peptide sequences of MG72 nucleases.

SEQ ID NO: 554 shows a PAM sequence compatible with an MG72 nuclease.

SEQ ID NO: 572 shows a nucleotide sequences of sgRNA engineered to function with an MG72 nuclease, where Ns denote nucleotides of a targeting sequence.

SEQ ID NO: 1649 shows the nucleotide sequence of an MG72 tracrRNA derived from the same loci as MG72 nucleases above.

### MG73

SEQ ID NOs: 296-305 and 1116-1118 show the full-length peptide sequences of MG73 nucleases.

SEQ ID NO: 555 shows a PAM sequence compatible with an MG73 nuclease.

SEQ ID NOs: 573-574 show nucleotide sequences of sgRNA engineered to function with an MG73 nucleases, where Ns denote nucleotides of a targeting sequence.

SEQ ID NOs: 1500-1505 show the peptide sequences of PAM-interacting domains of MG73 nucleases.

SEQ ID NOs: 1650-1651 show nucleotide sequences of MG73 tracrRNAs derived from the same loci as MG73 nucleases above.

### MG74

SEQ ID NOs: 306-355 and 1119-1160 show the full-length peptide sequences of MG74 nucleases. SEQ ID NO: 556 shows a PAM sequence compatible with an MG74 nuclease.

SEQ ID NO: 575 shows a nucleotide sequences of sgRNA engineered to function with an MG74 nuclease, where Ns denote nucleotides of a targeting sequence.

SEQ ID NOs: 1506-1519 show the peptide sequences of PAM-interacting domains of MG74 nucleases.

SEQ ID NO: 1652 shows the nucleotide sequence of an MG74 tracrRNA derived from the same loci as MG74 nucleases above.

### MG86

SEQ ID NOs: 356-402 and 1161-1206 show the full-length peptide sequences of MG86 nucleases.

SEQ ID NOs: 557-559 shows PAM sequences compatible with MG86 nucleases.

SEQ ID NOs: 576-577 show nucleotide sequences of sgRNA engineered to function with an MG86 nucleases, where Ns denote nucleotides of a targeting sequence.

SEQ ID NOs: 1520-1578 show the peptide sequences of PAM-interacting domains of MG86 nucleases.

SEQ ID NO: 1642 shows the nucleotide sequence of a single guide PAM of an MG86 nuclease.

SEQ ID NOs: 1653-1654 show nucleotide sequences of MG86 tracrRNAs derived from the same loci as MG86 nucleases above.

### MG87

SEQ ID NOs: 403-462 and 1207-1247 show the full-length peptide sequences of MG87 nucleases.

SEQ ID NOs: 560-562 shows PAM sequences compatible with MG87 nucleases.

SEQ ID NOs: 578-580 show nucleotide sequences of sgRNA engineered to function with an MG87 nucleases, where Ns denote nucleotides of a targeting sequence.

SEQ ID NOs: 1579-1615 show the peptide sequences of PAM-interacting domains of MG87 nucleases.

SEQ ID NOs: 1655-1657 show nucleotide sequences of MG87 tracrRNAs derived from the same loci as MG87 nucleases above.

### MG88

SEQ ID NOs: 463-482 and 1248-1258 show the full-length peptide sequences of MG88 nucleases.

SEQ ID NOs: 563-565 shows PAM sequences compatible with MG88 nucleases.

SEQ ID NOs: 581-583 show nucleotide sequences of sgRNA engineered to function with an MG88 nucleases, where Ns denote nucleotides of a targeting sequence.

SEQ ID NOs: 1616-1628 show the peptide sequences of PAM-interacting domains of MG88 nucleases.

SEQ ID NOs: 1658-1660 show nucleotide sequences of MG88 tracrRNAs derived from the same loci as MG88 nucleases above.

### MG89

SEQ ID NOs: 483-549 and 1259-1276 show the full-length peptide sequences of MG89 nucleases.

SEQ ID NOs: 566-567 shows PAM sequences compatible with MG89 nucleases.

SEQ ID NOs: 584-585 show nucleotide sequences of sgRNA engineered to function with an MG89 nucleases, where Ns denote nucleotides of a targeting sequence.

SEQ ID NOs: 1629-1641 show the peptide sequences of PAM-interacting domains of MG89 nucleases.

SEQ ID NOs: 1661-1662 show nucleotide sequences of MG88 tracrRNAs derived from the same loci as MG88 nucleases above.

### MG71-2 AAVS1 Targeting

SEQ ID NOs: 1663-1664 show the nucleotide sequences of sgRNAs engineered to function with an MG71-2 nuclease in order to target AAVS1.

SEQ ID NOs: 1665-1666 show the DNA sequences of AAVS1 target sites.

### MG73-1 TRAC Targeting

SEQ ID NO: 1667 shows the nucleotide sequence of an sgRNA engineered to function with an MG73-1 nuclease in order to target TRAC.

SEQ ID NO: 1668 shows the DNA sequence of a TRAC target site.

### MG89-2 TRAC Targeting

SEQ ID NOs: 1669-1675 show the nucleotide sequences of sgRNAs engineered to function with an MG89-2 nuclease in order to target TRAC. SEQ ID NOs: 1676-1682 show the DNA sequences of TRAC target sites.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The practice of some methods disclosed herein employ, unless otherwise indicated, techniques of immunology, biochemistry, chemistry, molecular biology, microbiology, cell biology, genomics, and recombinant DNA. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)) (which is entirely incorporated by reference herein).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

As used herein, a “cell” generally refers to a biological cell. A cell may be the basic structural, functional and/or biological unit of a living organism. A cell may originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g.,, Botryococcus braunii, Chlamydomonas reinhardtii, Nannochloropsis gaditana, Chlorella pyrenoidosa, Sargassum patens C. Agardh, and the like), seaweeds (e.g., kelp), a fungal cell (e.g.,, a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell).

The term “nucleotide,” as used herein, generally refers to a base-sugar-phosphate combination. A nucleotide may comprise a synthetic nucleotide. A nucleotide may comprise a synthetic nucleotide analog. Nucleotides may be monomeric units of a nucleic acid sequence (e.g., deoxyribonucleic acid (DNA) and ribonucleic acid (RNA)). The term nucleotide may include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives may include, for example, [αS]dATP, 7-deaza-dGTP and 7-deaza-dATP, and nucleotide derivatives that confer nuclease resistance on the nucleic acid molecule containing them. The term nucleotide as used herein may refer to dideoxyribonucleoside triphosphates (ddNTPs) and their derivatives. Illustrative examples of dideoxyribonucleoside triphosphates may include, but are not limited to, ddATP, ddCTP, ddGTP, ddITP, and ddTTP. A nucleotide may be unlabeled or detectably labeled, such as using moieties comprising optically detectable moieties (e.g., fluorophores). Labeling may also be carried out with quantum dots. Detectable labels may include, for example, radioactive isotopes, fluorescent labels, chemiluminescent labels, bioluminescent labels, and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g., biotin-11-dUTP, biotin-16-dUTP, biotin-20-dUTP).

The terms “polynucleotide,” “oligonucleotide,” and “nucleic acid” are used interchangeably to generally refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure and may perform any function. A polynucleotide may comprise one or more analogs (e.g., altered backbone, sugar, or nucleobase). If present, modifications to the nucleotide structure may be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides may be interrupted by non-nucleotide components.

The terms “transfection” or “transfected” generally refer to introduction of a nucleic acid into a cell by non-viral or viral-based methods. The nucleic acid molecules may be gene sequences encoding complete proteins or functional portions thereof. See, e.g., Sambrook et al., 1989, Molecular Cloning: A Laboratory Manual, 18.1-18.88.

The terms “peptide,” “polypeptide,” and “protein” are used interchangeably herein to generally refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer may be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids may include natural amino acids and non-natural amino acids, which have been chemically modified to include a group or a chemical moiety not naturally present on the amino acid. Amino acid analogues may refer to amino acid derivatives. The term “amino acid” includes both D-amino acids and L-amino acids.

As used herein, the “non-native” can generally refer to a nucleic acid or polypeptide sequence that is not found in a native nucleic acid or protein. Non-native may refer to affinity tags. Non-native may refer to fusions. Non-native may refer to a naturally occurring nucleic acid or polypeptide sequence that comprises mutations, insertions and/or deletions. A non-native sequence may exhibit and/or encode for an activity (e.g., enzymatic activity, methyltransferase activity, acetyltransferase activity, kinase activity, ubiquitinating activity, etc.) that may also be exhibited by the nucleic acid and/or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid and/or polypeptide sequence encoding a chimeric nucleic acid and/or polypeptide.

The term “promoter”, as used herein, generally refers to the regulatory DNA region which controls transcription or expression of a gene and which may be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. A ‘basal promoter’, also referred to as a ‘core promoter’, may generally refer to a promoter that contains all the basic necessary elements to promote transcriptional expression of an operably linked polynucleotide. Eukaryotic basal promoters typically, though not necessarily, contain a TATA-box and/or a CAAT box.

The term “expression”, as used herein, generally refers to the process by which a nucleic acid sequence or a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, “operably linked”, “operable linkage”, “operatively linked”, or grammatical equivalents thereof generally refer to juxtaposition of genetic elements, e.g., a promoter, an enhancer, a polyadenylation sequence, etc., wherein the elements are in a relationship permitting them to operate in the expected manner For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

A “vector” as used herein, generally refers to a macromolecule or association of macromolecules that comprises or associates with a polynucleotide and which may be used to mediate delivery of the polynucleotide to a cell. Examples of vectors include plasmids, viral vectors, liposomes, and other gene delivery vehicles. The vector generally comprises genetic elements, e.g., regulatory elements, operatively linked to a gene to facilitate expression of the gene in a target.

As used herein, “an expression cassette” and “a nucleic acid cassette” are used interchangeably generally to refer to a combination of nucleic acid sequences or elements that are expressed together or are operably linked for expression. In some cases, an expression cassette refers to the combination of regulatory elements and a gene or genes to which they are operably linked for expression.

A “functional fragment” of a DNA or protein sequence generally refers to a fragment that retains a biological activity (either functional or structural) that is substantially similar to a biological activity of the full-length DNA or protein sequence. A biological activity of a DNA sequence may be its ability to influence expression in a manner known to be attributed to the full-length sequence.

As used herein, an “engineered” object generally indicates that the object has been modified by human intervention. According to non-limiting examples: a nucleic acid may be modified by changing its sequence to a sequence that does not occur in nature; a nucleic acid may be modified by ligating it to a nucleic acid that it does not associate with in nature such that the ligated product possesses a function not present in the original nucleic acid; an engineered nucleic acid may synthesized in vitro with a sequence that does not exist in nature; a protein may be modified by changing its amino acid sequence to a sequence that does not exist in nature; an engineered protein may acquire a new function or property. An “engineered” system comprises at least one engineered component.

As used herein, “synthetic” and “artificial” are used interchangeably to refer to a protein or a domain thereof that has low sequence identity (e.g., less than 50% sequence identity, less than 25% sequence identity, less than 10% sequence identity, less than 5% sequence identity, less than 1% sequence identity) to a naturally occurring human protein. For example, VPR and VP64 domains are synthetic transactivation domains.

The term “tracrRNA” or “tracr sequence”, as used herein, can generally refer to a nucleic acid with at least about 5%, 10%, 20%, 30%, 40%, 50%, 60%, 70%, 80%, 90%, 95%, or 100% sequence identity and/or sequence similarity to a wild type exemplary tracrRNA sequence (e.g., a tracrRNA from S. pyogenes S. aureus, etc.). tracrRNA can refer to a nucleic acid with at most about 5%, 10%, 20%, 30%, 40%, 50%, 60%, 70%, 80%, 90%, or 100% sequence identity and/or sequence similarity to a wild type exemplary tracrRNA sequence (e.g., a tracrRNA from S. pyogenes S. aureus, etc). tracrRNA may refer to a modified form of a tracrRNA that can comprise a nucleotide change such as a deletion, insertion, or substitution, variant, mutation, or chimera. A tracrRNA may refer to a nucleic acid that can be at least about 60% identical to a wild type exemplary tracrRNA (e.g., a tracrRNA from S. pyogenes S. aureus, etc) sequence over a stretch of at least 6 contiguous nucleotides. For example, a tracrRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical to a wild type exemplary tracrRNA (e.g., a tracrRNA from S. pyogenes S. aureus, etc) sequence over a stretch of at least 6 contiguous nucleotides. Type II tracrRNA sequences can be predicted on a genome sequence by identifying regions with complementarity to part of the repeat sequence in an adjacent CRISPR array.

As used herein, a “guide nucleic acid” can generally refer to a nucleic acid that may hybridize to another nucleic acid. A guide nucleic acid may be RNA. A guide nucleic acid may be DNA. The guide nucleic acid may be programmed to bind to a sequence of nucleic acid site-specifically. The nucleic acid to be targeted, or the target nucleic acid, may comprise nucleotides. The guide nucleic acid may comprise nucleotides. A portion of the target nucleic acid may be complementary to a portion of the guide nucleic acid. The strand of a double-stranded target polynucleotide that is complementary to and hybridizes with the guide nucleic acid may be called the complementary strand. The strand of the double-stranded target polynucleotide that is complementary to the complementary strand, and therefore may not be complementary to the guide nucleic acid may be called noncomplementary strand. A guide nucleic acid may comprise a polynucleotide chain and can be called a “single guide nucleic acid.” A guide nucleic acid may comprise two polynucleotide chains and may be called a “double guide nucleic acid.” If not otherwise specified, the term “guide nucleic acid” may be inclusive, referring to both single guide nucleic acids and double guide nucleic acids. A guide nucleic acid may comprise a segment that can be referred to as a “nucleic acid-targeting segment” or a “nucleic acid-targeting sequence.” A nucleic acid-targeting segment may comprise a sub-segment that may be referred to as a “protein binding segment” or “protein binding sequence” or “Cas protein binding segment”.

The term “sequence identity” or “percent identity” in the context of two or more nucleic acids or polypeptide sequences, generally refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation (E) of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation (E) of 1000000, and the PAM30 scoring matrix setting gap costs at 9 to open gaps and 1 to extend gaps for sequences of less than 30 residues (these are the default parameters for BLASTP in the BLAST suite available at https://blast.ncbi.nlm nih.gov); CLUSTALW with parameters of ; the Smith-Waterman homology search algorithm with parameters of a match of 2, a mismatch of -1, and a gap of -1; MUSCLE with default parameters; MAFFT with parameters retree of 2 and maxiterations of 1000; Novafold with default parameters; HMMER hmmalign with default parameters.

As used herein, the term “RuvC III domain” generally refers to a third discontinuous segment of a RuvC endonuclease domain (the RuvC nuclease domain being comprised of three discontiguous segments, RuvC_I, RuvC_II, and RuvC_III). A RuvC domain or segments thereof can generally be identified by alignment to known domain sequences, structural alignment to proteins with annotated domains, or by comparison to Hidden Markov Models (HMMs) built based on known domain sequences (e.g., Pfam HMM PF18541 for RuvC_III).

As used herein, the term “HNH domain” generally refers to an endonuclease domain having characteristic histidine and asparagine residues. An HNH domain can generally be identified by alignment to known domain sequences, structural alignment to proteins with annotated domains, or by comparison to Hidden Markov Models (HMMs) built based on known domain sequences (e.g., Pfam HMM PF01844 for domain HNH).

As used herein, the term “Wedge” (WED) domain generally refers to a domain (e.g. present in a Cas protein) interacting primarily with repeat:anti-repeat duplex of the sgRNA and PAM duplex.

As used herein, the term “PAM interacting domain” generally refers to a domain interacting with the protospacer-adjacent motif (PAM) external to the seed sequence in a region targeted by a Cas protein. Examples of PAM-interacting domains include, but are not limited to, Topoisomerase-homology (TOPO) domains and C-terminal domains (CTD) present in Cas proteins.

Included in the current disclosure are variants of any of the enzymes described herein with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and R chain length for one another. Additionally, or alternatively, by comparing aligned sequences of homologous proteins from different species, conservative substitutions can be identified by locating amino acid residues that have been mutated between species (e.g., non-conserved residues) without altering the basic functions of the encoded proteins. Such conservatively substituted variants may include variants with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% identity to any one of the endonuclease protein sequences described herein (e.g. MG44, MG46, MG71, MG72, MG73, MG74, MG86, MG87, MG88, or MG89 family endonucleases described herein, or any other family nuclease described herein). In some embodiments, such conservatively substituted variants are functional variants. Such functional variants can encompass sequences with substitutions such that the activity of one or more critical active site residues or guide RNA binding residues of the endonuclease are not disrupted. In some embodiments, a functional variant of any of the proteins described herein lacks substitution of at least one of the conserved or functional residues of RuvC or HNH domains of endonucleases described herein. In some embodiments, a functional variant of any of the proteins described herein lacks substitution of all of the conserved or functional residues of the RuvC or HNH domains of endonucleases described herein.

Conservative substitution tables providing functionally similar amino acids are available from a variety of references (see, for e.g., Creighton, Proteins: Structures and Molecular Properties (W H Freeman & Co.; 2nd edition (December 1993)). The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)

### Overview

The discovery of new Cas enzymes with unique functionality and structure may offer the potential to further disrupt deoxyribonucleic acid (DNA) editing technologies, improving speed, specificity, functionality, and ease of use. Relative to the predicted prevalence of Clustered Regularly Interspaced Short Palindromic Repeats (CRISPR) systems in microbes and the sheer diversity of microbial species, relatively few functionally characterized CRISPR/Cas enzymes exist in the literature. This is partly because a huge number of microbial species may not be readily cultivated in laboratory conditions. Metagenomic sequencing from natural environmental niches that represent large numbers of microbial species may offer the potential to drastically increase the number of new CRISPR/Cas systems known and speed the discovery of new oligonucleotide editing functionalities. A recent example of the fruitfulness of such an approach is demonstrated by the 2016 discovery of CasX/CasY CRISPR systems from metagenomic analysis of natural microbial communities.

CRISPR/Cas systems are RNA-directed nuclease complexes that have been described to function as an adaptive immune system in microbes. In their natural context, CRISPR/Cas systems occur in CRISPR (clustered regularly interspaced short palindromic repeats) operons or loci, which generally comprise two parts: (i) an array of short repetitive sequences (30-40 bp) separated by equally short spacer sequences, which encode the RNA-based targeting element; and (ii) ORFs encoding the Cas encoding the nuclease polypeptide directed by the RNA-based targeting element alongside accessory proteins/enzymes. Efficient nuclease targeting of a particular target nucleic acid sequence generally requires both (i) complementary hybridization between the first 6-8 nucleic acids of the target (the target seed) and the crRNA guide; and (ii) the presence of a protospacer-adjacent motif (PAM) sequence within a defined vicinity of the target seed (the PAM usually being a sequence not commonly represented within the host genome). Depending on the exact function and organization of the system, CRISPR-Cas systems are commonly organized into 2 classes, 5 types and 16 subtypes based on shared functional characteristics and evolutionary similarity.

Class I CRISPR-Cas systems have large, multisubunit effector complexes, and comprise Types I, III, and IV.

Type I CRISPR-Cas systems are considered of moderate complexity in terms of components. In Type I CRISPR-Cas systems, the array of RNA-targeting elements is transcribed as a long precursor crRNA (pre-crRNA) that is processed at repeat elements to liberate short, mature crRNAs that direct the nuclease complex to nucleic acid targets when they are followed by a suitable short consensus sequence called a protospacer-adjacent motif (PAM). This processing occurs via an endoribonuclease subunit (Cas6) of a large endonuclease complex called Cascade, which also comprises a nuclease (Cas3) protein component of the crRNA-directed nuclease complex. Cas I nucleases function primarily as DNA nucleases.

Type III CRISPR systems may be characterized by the presence of a central nuclease, known as Cas10, alongside a repeat-associated mysterious protein (RAMP) that comprises Csm or Cmr protein subunits. Like in Type I systems, the mature crRNA is processed from a pre-crRNA using a Cas6-like enzyme. Unlike type I and II systems, type III systems appear to target and cleave DNA-RNA duplexes (such as DNA strands being used as templates for an RNA polymerase).

Type IV CRISPR-Cas systems possess an effector complex that consists of a highly reduced large subunit nuclease (csf1), two genes for RAMP proteins of the Cas5 (csf3) and Cas7 (csf2) groups, and, in some cases, a gene for a predicted small subunit; such systems are commonly found on endogenous plasmids.

Class II CRISPR-Cas systems generally have single-polypeptide multidomain nuclease effectors, and comprise Types II, V and VI.

Type II CRISPR-Cas systems are considered the simplest in terms of components. In Type II CRISPR-Cas systems, the processing of the CRISPR array into mature crRNAs does not require the presence of a special endonuclease subunit, but rather a small trans-encoded crRNA (tracrRNA) with a region complementary to the array repeat sequence; the tracrRNA interacts with both its corresponding effector nuclease (e.g. Cas9) and the repeat sequence to form a precursor dsRNA structure, which is cleaved by endogenous RNAse III to generate a mature effector enzyme loaded with both tracrRNA and crRNA. Cas II nucleases are known as DNA nucleases. Type 2 effectors generally exhibit a structure consisting of a RuvC-like endonuclease domain that adopts the RNase H fold with an unrelated HNH nuclease domain inserted within the folds of the RuvC-like nuclease domain The RuvC-like domain is responsible for the cleavage of the target (e.g., crRNA-complementary) DNA strand, while the HNH domain is responsible for cleavage of the displaced DNA strand. Type II effectors also can comprise PAM-interacting or PI domains that comprise TOPO and CTD regions that contribute to recognition of a protospacer adjacent motif (PAM) site in the vicinity of the crRNA-targeted DNA region.

Type V CRISPR-Cas systems are characterized by a nuclease effector (e.g. Cas12) structure similar to that of Type II effectors, comprising a RuvC-like domain Similar to Type II, most (but not all) Type V CRISPR systems use a tracrRNA to process pre-crRNAs into mature crRNAs; however, unlike Type II systems which requires RNAse III to cleave the pre-crRNA into multiple crRNAs, type V systems are capable of using the effector nuclease itself to cleave pre-crRNAs Like Type-II CRISPR-Cas systems, Type V CRISPR-Cas systems are again known as DNA nucleases. Unlike Type II CRISPR-Cas systems, some Type V enzymes (e.g., Cas12a) appear to have a robust single-stranded nonspecific deoxyribonuclease activity that is activated by the first crRNA directed cleavage of a double-stranded target sequence.

Type VI CRIPSR-Cas systems have RNA-guided RNA endonucleases. Instead of RuvC-like domains, the single polypeptide effector of Type VI systems (e.g. Cas13) comprises two HEPN ribonuclease domains. Differing from both Type II and V systems, Type VI systems also appear to not need a tracrRNA for processing of pre-crRNA into crRNA. Similar to type V systems, however, some Type VI systems (e.g., C2C2) appear to possess robust single-stranded nonspecific nuclease (ribonuclease) activity activated by the first crRNA directed cleavage of a target RNA.

Because of their simpler architecture, Class II CRISPR-Cas have been most widely adopted for engineering and development as designer nuclease/genome editing applications.

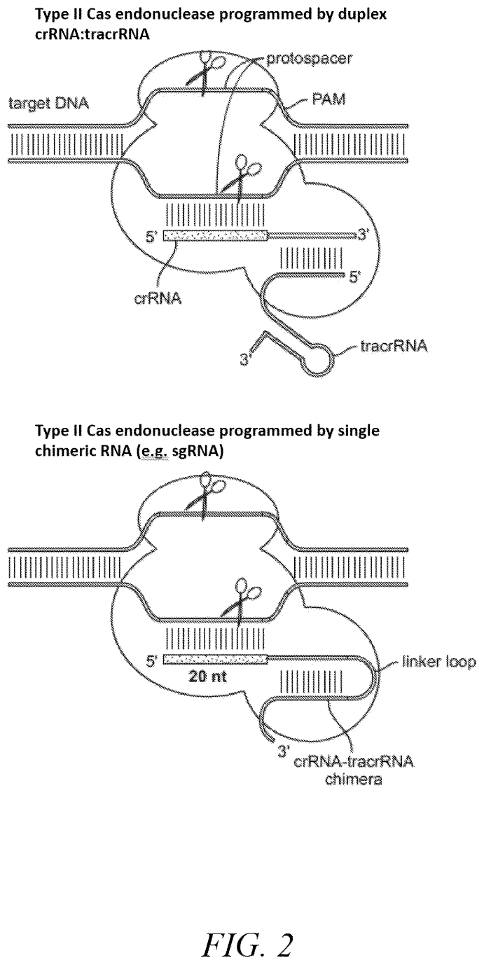

One of the early adaptations of such a system for in vitro use can be found in Jinek et al. (Science. 2012 Aug. 17; 337(6096):816-21, which is entirely incorporated herein by reference). The Jinek study first described a system that involved (i) recombinantly-expressed, purified full-length Cas9 (e.g., a Class II, Type II Cas enzyme) isolated from S. pyogenes SF370, (ii) purified mature ˜42 nt crRNA bearing a ˜20 nt 5′ sequence complementary to the target DNA sequence desired to be cleaved followed by a 3′ tracr-binding sequence (the whole crRNA being in vitro transcribed from a synthetic DNA template carrying a T7 promoter sequence); (iii) purified tracrRNA in vitro transcribed from a synthetic DNA template carrying a T7 promoter sequence, and (iv) Mg²⁺. Jinek later described an improved, engineered system wherein the crRNA of (ii) is joined to the 5′ end of (iii) by a linker (e.g., GAAA) to form a single fused synthetic guide RNA (sgRNA) capable of directing Cas9 to a target by itself (compare top and bottom panel of FIG. 2).

Mali et al. (Science. 2013 Feb. 15; 339(6121): 823-826.), which is entirely incorporated herein by reference, later adapted this system for use in mammalian cells by providing DNA vectors encoding (i) an ORF encoding codon-optimized Cas9 (e.g., a Class II, Type II Cas enzyme) under a suitable mammalian promoter with a C-terminal nuclear localization sequence (e.g., SV40 NLS) and a suitable polyadenylation signal (e.g., TK pA signal); and (ii) an ORF encoding an sgRNA (having a 5′ sequence beginning with G followed by 20 nt of a complementary targeting nucleic acid sequence joined to a 3′ tracr-binding sequence, a linker, and the tracrRNA sequence) under a suitable Polymerase III promoter (e.g., the U6 promoter) .

### MG Enzymes

In one aspect, the present disclosure provides for an engineered nuclease system comprising (a) an endonuclease. In some cases, the endonuclease is a Cas endonuclease. In some cases, the endonuclease is a Type II, Class II Cas endonuclease. The endonuclease may comprise a RuvC domain or a portion thereof (e.g. a RuvC_I, RuvC_II, or RuvC_III domain) The endonuclease may comprise an HNH domain The endonuclease may comprise a PAM-interacting (PI) domain.

In some cases, the endonuclease may comprise a variant having at least about 30%, at least about 35%, at least about 40%, at least about 45%, at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1-549 or 602-1276. In some cases, the endonuclease may be substantially identical to any one of SEQ ID NOs: 1-549 or 602-1276.

In some embodiments, an endonuclease system according to the present disclosure can comprise any of the components describe in Table 1.

TABLE 1
PAM sequence specificities and related data for MG enzymes

| | Enzyme | tracrRNA | sgRNA | | | 3′ PAM |
|---|---|---|---|---|---|---|
| | protein | SEQ | SEQ | | | SEQ |
| | SEQ ID | ID | ID | PI | | ID |
| Enzyme | NO | NO: | NO: | domain | 3′ PAM | NO: |
| MG44-1 | 1 | 1645 | 568 | 1277 | nnnAACnn | 550 |
| MG46-1 | 217 | 1646 | 569 | 1420 | nnYAAnnn | 551 |
| MG71-1 | 258 | 1647 | 570, | 1498 | nnRMYnn | 552 |
| | | | 1643 | | | |
| MG71-2 | 259 | 1648 | 571 | 1683 | nnnACTnn | 553 |
| MG72-1 | 284 | 1649 | 572 | 1428 | nnWAnCnn | 554 |
| MG73-1 | 296 | 1650 | 573 | 1500 | nnRnTTnn | 555 |
| MG73-2 | 297 | 1651 | 574 | 1501 | nnRnTTnn | 555 |
| MG74-1 | 306 | 1652 | 575 | 1506 | nnnnRMAA | 556 |
| MG86-2 | 357 | 1654 | 577 | 1521 | nRAAR, | 558, 559 |
| | | | | | nYAAAA | |
| MG87-1 | 403 | 1655 | 578 | 1579 | nnnnGTY | 560 |
| MG87-2 | 404 | 1656 | 579 | 1580 | nnRnG | 561 |
| MG87-3 | 405 | 1657 | 580 | 1581 | nnrtRTY | 562 |
| MG88-1 | 463 | 1658 | 581 | 1616 | nRRRa | 563 |
| MG88-2 | 464 | 1659 | 582 | 1626 | nRRwww | 564 |
| MG88-3 | 465 | 1660 | 583 | 1617 | nRRRnY | 565 |
| MG86-1 | 356 | 1653 | 576 | 1520 | nyAAAr | 557 |
| MG89-2 | 484 | 1661 | 584 | 1633 | nnnnCC | 566 |

In some cases, the endonuclease may comprise a variant having one or more nuclear localization sequences (NLSs). The NLS may be proximal to the N- or C-terminus of said endonuclease. The NLS may be appended N-terminal or C-terminal to any one of SEQ ID NOs: 1-549 or 602-1276, or to a variant having at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% identity to any one of SEQ ID NOs: 1-549 or 602-1276. The NLS may be an SV40 large T antigen NLS. The NLS may be a c-myc NLS. The NLS can comprise a sequence with at least about 80%, at least about 85%, at least about 90%, at least about 95%, at least about 99% identity to any one of SEQ ID NOs: 586-601. The NLS can comprise a sequence substantially identical to any one of SEQ ID NOs: 586-601. The NLS can comprise any of the sequences in Table 2 below, or a combination thereof:

TABLE 2
Example NLS Sequences that can be used
with Cas Effectors According to the
Disclosure

| | | SEQ |
|---|---|---|
| | NLS amino | ID |
| Source | acid sequence | NO: |
| SV40 | PKKKRKV | 586 |
| nucleoplasmin | KRPAATKKAGQA | 587 |
| bipartite NLS | KKKK | |
| c-myc NLS | PAAKRVKLD | 588 |
| c-myc NLS | RQRRNELKRSP | 589 |
| bRNPAI M9 NLS | NQSSNFGPMKGGNF | 590 |
| | GGRSSGPYGGGGQY | |
| | FAKPRNQGGY | |
| Importin-alpha | RMRIZFKNKGKDTA | 591 |
| IBB domain | ELRRRRVEVSVELR | |
| | KAKKDEQILKRRNV | |
| Myoma T protein | VSRKRPRP | 592 |
| Myoma T protein | PPKKARED | 593 |
| p53 | PQPKKKPL | 594 |
| mouse c-ab1 IV | SALIKKKKKMAP | 595 |
| influenza | DRLRR | 596 |
| virus NS1 | | |
| influenza | PKQKKRK | 597 |
| virus NS1 | | |
| Hepatitis virus | RKLKKKIKKL | 598 |
| delta antigen | | |
| mouse Mx1 | REKKKFLKRR | 599 |
| protein | | |
| human poly | KRKGDEVDGVDEV | 600 |
| (ADP-ribose) | | |
| polymerase | AKKKSKK | |
| steroid hormone | RKCLQAGMNLEAR | 601 |
| receptor (human) | KTKK | |
| glucocorticoid | | |

In one aspect, the present disclosure provides for engineered nuclease system comprising: (a) an endonuclease configured to have selectivity for a protospacer adjacent motif (PAM) sequence comprising any one of SEQ ID NOs: 550-567, wherein the endonuclease is a class 2, type II Cas endonuclease. In some embodiments, the system further comprises (b) an engineered guide nucleic acid structure configured to form a complex with the endonuclease comprising: (i) a targeting nucleic acid sequence configured to hybridize to a target deoxyribonucleic acid sequence; and (ii) a tracr ribonucleic acid sequence configured to bind to the endonuclease. In some embodiments, the endonuclease is derived from an uncultivated microorganism. In some embodiments, the endonuclease has not been engineered to bind to a different PAM sequence than a native PAM sequence of the endonuclease. In some embodiments, the endonuclease is not a Cas9 endonuclease, a Cas14 endonuclease, a Cas12a endonuclease, a Cas12b endonuclease, a Cas 12c endonuclease, a Cas12d endonuclease, a Cas12e endonuclease, a Cas13a endonuclease, a Cas13b endonuclease, a Cas13c endonuclease, or a Cas 13d endonuclease. In some embodiments, the endonuclease has less than 80% identity to a Cas9 endonuclease. In some embodiments, the endonuclease further comprises a PI domain comprising a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1277-1641 or 1683, or a variant thereof. In some embodiments, the endonuclease further comprises a PI domain having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to a PI domain of any one of SEQ ID NOs: 1-549 or 602-1276. In some embodiments, the endonuclease further comprises a PI domain comprising a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1, 217, 258, 259, 284, 296, 297, 306, 357, 403, 404, 405, 463, 464, 465, 356, or 484, or a variant thereof. In some embodiments, the endonuclease further comprises a PI domain comprising a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 259, 296, or 484, or a variant thereof. In some embodiments, the endonuclease further comprises a RuvC domain In some embodiments, the RuvC domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to a RuvC domain of any one of SEQ ID NOs: 1, 217, 258, 259, 284, 296, 297, 306, 357, 403, 404, 405, 463, 464, 465, 356, or 484, or a variant thereof. In some embodiments, the RuvC domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to a RuvC domain of any one of SEQ ID NOs: 259, 296, or 484, or a variant thereof. In some embodiments, the endonuclease further comprises an HNH domain In some embodiments, the HNH domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to an HNH domain of any one of SEQ ID NOs: 1, 217, 258, 259, 284, 296, 297, 306, 357, 403, 404, 405, 463, 464, 465, 356, or 484, or a variant thereof. In some embodiments, the HNH domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to an HNH domain of any one of SEQ ID NOs: 259, 296, or 484, or a variant thereof. In some embodiments, the endonuclease is configured to have selectivity for a PAM sequence comprising any one of SEQ ID NOs: 553, 555, or 566, or a variant thereof. In some embodiments, the engineered guide nucleic acid structure comprises at least two ribonucleic acid polynucleotides. In some embodiments, the engineered guide nucleic acid structure comprises one ribonucleic acid polynucleotide comprising the guide ribonucleic acid sequence and the tracr ribonucleic acid sequence. In some embodiments, the engineered guide nucleic acid structure comprises a tracr sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1645-1662 or wherein the engineered guide nucleic acid structure comprises a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to non-degenerate nucleotides of any one of SEQ ID NOs: 568-585 or 1643-1644. In some embodiments, the engineered guide nucleic acid structure comprises a tracr sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1648, 1650, or 1661 or wherein the engineered guide nucleic acid structure comprises a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to non-degenerate nucleotides of any one of SEQ ID NOs: 571, 573, or 584. In some embodiments, the targeting nucleic acid sequence is complementary to a prokaryotic, bacterial, archaeal, eukaryotic, fungal, plant, mammalian, or human genomic sequence. In some embodiments, the targeting nucleic acid sequence is 15-24 nucleotides in length. In some embodiments, the endonuclease comprises one or more nuclear localization sequences (NLSs) proximal to an N- or C-terminus of the endonuclease. the NLS comprises a sequence comprising any one of SEQ ID NOs: 586-601, or a variant thereof. In some embodiments, the system further comprises a single- or double-stranded DNA repair template comprising from 5′ to 3′: a first homology arm comprising a sequence of at least 20 nucleotides 5′ to the target deoxyribonucleic acid sequence, a synthetic DNA sequence of at least 10 nucleotides, and a second homology arm comprising a sequence of at least 20 nucleotides 3′ to the target sequence. In some embodiments, the first or second homology arm comprises a sequence of at least 40, 80, 120, 150, 200, 300, 500, or 1,000 nucleotides. In some embodiments, the system further comprises a source of Mg²⁺. In some embodiments, the endonuclease and the tracr ribonucleic acid sequence are derived from distinct bacterial species within a same phylum. In some embodiments, the endonuclease comprises any one of SEQ ID NOs: 1, 217, 258, 259, 284, 296, 297, 306, 357, 403, 404, 405, 463, 464, 465, 356, or 484: or a variant thereof having at least 55% identity thereto. In some embodiments, the sequence identity is determined by a BLASTP, CLUSTALW, MUSCLE, MAFFT algorithm, or a CLUSTALW algorithm with the Smith-Waterman homology search algorithm parameters. In some embodiments, the sequence identity is determined by the BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment. In some embodiments, the PAM sequence is 3′ to the target deoxyribonucleic acid sequence

In some aspects, the present disclosure provides for a nucleic acid comprising an engineered nucleic acid sequence optimized for expression in an organism, wherein the nucleic acid encodes a class 2, type II Cas endonuclease configured to be selective for a protospacer adjacent motif (PAM) comprising any one of SEQ ID NOs: 550-567. In some embodiments, the endonuclease further comprises a PI domain comprising a sequence having at least at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1277-1641 or 1683, or a variant thereof, or wherein the endonuclease further comprises a PI domain having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to a PI domain of any one of SEQ ID NOs: 1-549 or 602-1276. In some embodiments, the endonuclease further comprises a PI domain comprising a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1, 217, 258, 259, 284, 296, 297, 306, 357, 403, 404, 405, 463, 464, 465, 356, or 484, or a variant thereof. In some embodiments, the endonuclease further comprises a PI domain comprising a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 259, 296, or 484, or a variant thereof. In some embodiments, the endonuclease further comprises a RuvC domain. In some embodiments, the RuvC domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to a RuvC domain of any one of SEQ ID NOs: 1, 217, 258, 259, 284, 296, 297, 306, 357, 403, 404, 405, 463, 464, 465, 356, or 484, or a variant thereof. In some embodiments, the RuvC domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to a RuvC domain of any one of SEQ ID NOs: 259, 296, or 484, or a variant thereof. In some embodiments, the endonuclease further comprises an HNH domain. In some embodiments, the HNH domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to an HNH domain of any one of SEQ ID NOs: 1, 217, 258, 259, 284, 296, 297, 306, 357, 403, 404, 405, 463, 464, 465, 356, or 484, or a variant thereof. In some embodiments, the HNH domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to an HNH domain of any one of SEQ ID NOs: 259, 296, or 484, or a variant thereof. In some embodiments, the endonuclease comprises any one of SEQ ID NOs: 1, 217, 258, 259, 284, 296, 297, 306, 357, 403, 404, 405, 463, 464, 465, 356, or 484, or a variant thereof having at least 55%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity thereto. In some embodiments, the organism is a bacterial, archaeal, eukaryotic, fungal, plant, mammalian, or human organism,

In some aspects, the present disclosure provides for an engineered nuclease system comprising: (a) an endonuclease having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1-549, 602-1276, or a variant thereof; and (b) an engineered guide nucleic acid structure, wherein the engineered guide RNA is configured to form a complex with the endonuclease and the engineered guide RNA comprises a targeting nucleic acid sequence configured to hybridize to a target nucleic acid sequence. In some embodiments, the endonuclease comprises any one of SEQ ID NOs: 1, 217, 258, 259, 284, 296, 297, 306, 357, 403, 404, 405, 463, 464, 465, 356, or 484, or a variant thereof having at least 55% identity, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity thereto. In some embodiments, the endonuclease further comprises a RuvC domain In some embodiments, the RuvC domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to a RuvC domain of any one of SEQ ID NOs: 1, 217, 258, 259, 284, 296, 297, 306, 357, 403, 404, 405, 463, 464, 465, 356, or 484, or a variant thereof. In some embodiments, the RuvC domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to a RuvC domain of any one of SEQ ID NOs: 259, 296, or 484, or a variant thereof In some embodiments, the endonuclease further comprises an HNH domain In some embodiments, the HNH domain at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to an HNH domain of any one of SEQ ID NOs: 1, 217, 258, 259, 284, 296, 297, 306, 357, 403, 404, 405, 463, 464, 465, 356, or 484, or a variant thereof. In some embodiments, the HNH domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to an HNH domain of any one of SEQ ID NOs: 259, 296, or 484, or a variant thereof. In some embodiments, the engineered guide nucleic acid structure comprises a tracr sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1645-1662 or wherein the engineered guide nucleic acid structure comprises a sequence having at least 80% identity to non-degenerate nucleotides of any one of SEQ ID NOs: 568-585 or 1643-1644. In some embodiments, the engineered guide nucleic acid structure comprises a tracr sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1648, 1650, or 1661 or wherein the engineered guide nucleic acid structure comprises a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to non-degenerate nucleotides of any one of SEQ ID NOs: 571, 573, or 584. In some embodiments, the engineered guide nucleic acid structure is configured to form a complex with the endonuclease and comprises (i) a targeting nucleic acid sequence configured to hybridize to a target deoxyribonucleic acid sequence; and (ii) a tracr ribonucleic acid sequence configured to bind to the endonuclease. In some embodiments, the targeting nucleic acid sequence is complementary to a bacterial, archaeal, eukaryotic, fungal, plant, mammalian, or human genomic sequence. In some embodiments, the targeting nucleic acid sequence is 15-24 nucleotides in length.

In some aspects, the present disclosure provides for an engineered nuclease system comprising: (a) an engineered guide nucleic acid structure comprising: (i) a tracr sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1645-1662; or (ii) a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to non- degenerate nucleotides of any one of SEQ ID NOs: 568-585 or 1643-1644, and (b) a class 2, type II Cas endonuclease configured to bind to the engineered guide nucleic acid structure. In some embodiments, the engineered guide nucleic acid structure comprises a tracr sequence having at least 80% identity to any one of SEQ ID NOs: 1648, 1650, or 1661 or wherein the engineered guide nucleic acid structure comprises a sequence having at least 80% identity to non-degenerate nucleotides of any one of SEQ ID NOs: 571, 573, or 584. In some embodiments, the engineered guide nucleic acid structure is configured to form a complex with the endonuclease and comprises (i) a targeting nucleic acid sequence configured to hybridize to a target deoxyribonucleic acid sequence; and (ii) a tracr ribonucleic acid sequence configured to bind to the endonuclease. In some embodiments, the targeting nucleic acid sequence is complementary to a bacterial, archaeal, eukaryotic, fungal, plant, mammalian, or human genomic sequence. In some embodiments, the targeting nucleic acid sequence is 15-24 nucleotides in length. In some embodiments, the endonuclease comprises a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1-549, 602-1276, or a variant thereof. In some embodiments, the endonuclease comprises a sequence according to any one of SEQ ID NOs: 1, 217, 258, 259, 284, 296, 297, 306, 357, 403, 404, 405, 463, 464, 465, 356, or 484, or a variant thereof having at least 55%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity thereto.

In some aspects, the present disclosure provides for an engineered guide nucleic acid structure comprising: (a) a targeting nucleic acid sequence comprising a nucleotide sequence that is complementary to a target sequence in a target DNA molecule; and (b) a protein-binding segment comprising two complementary stretches of nucleotides that hybridize to form a double-stranded RNA (dsRNA) duplex, one of which comprising a tracr sequence, wherein the two complementary stretches of nucleotides are covalently linked to one another with intervening nucleotides, and wherein the engineered guide ribonucleic acid polynucleotide is capable of forming a complex with an endonuclease having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1-549, 602-1276, or a variant thereof, and targeting the complex to the target sequence of the target DNA molecule. In some embodiments, the endonuclease comprises any one of SEQ ID NOs: 1, 217, 258, 259, 284, 296, 297, 306, 357, 403, 404, 405, 463, 464, 465, 356, or 484, or a variant thereof having at least 55%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity thereto. In some embodiments, the targeting nucleic acid sequence is complementary to a bacterial, archaeal, eukaryotic, fungal, plant, mammalian, or human genomic sequence. In some embodiments, the targeting nucleic acid sequence is 15-24 nucleotides in length. In some embodiments, the engineered guide nucleic acid structure comprises a tracr sequence having at least 80% identity to any one of SEQ ID NOs: 1645-1662 or wherein the engineered guide nucleic acid structure comprises a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to non- degenerate nucleotides of any one of SEQ ID NOs: 568-585 or 1643-1644. In some embodiments, the engineered guide nucleic acid structure comprises a tracr sequence having at least 80% identity to any one of SEQ ID NOs: 1648, 1650, or 1661 or wherein the engineered guide nucleic acid structure comprises a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to non-degenerate nucleotides of any one of SEQ ID NOs: 571, 573, or 584.

In some aspects, the present disclosure provides for an engineered vector comprising any of the nucleic acids described herein. In some embodiments, the vector is a plasmid, a minicircle, a CELiD, an adeno-associated virus (AAV) derived virion, a lentivirus, or an adenovirus

In some aspects, the present disclosure provides for a cell comprising any of the vectors described herein or any of the nucleic acids described herein. In some embodiments, the cell is a bacterial, archaeal, eukaryotic, fungal, plant, mammalian, or human cell.

In some aspects, the present disclosure provides for a method of manufacturing an endonuclease, comprising cultivating any of the cells described herein.

In some aspects, the present disclosure provides for a method for binding, cleaving, marking, or modifying a double-stranded deoxyribonucleic acid polynucleotide, comprising: contacting the double-stranded deoxyribonucleic acid polynucleotide with a class 2, type II Cas endonuclease in complex with an engineered guide nucleic acid structure configured to bind to the endonuclease and the double-stranded deoxyribonucleic acid polynucleotide; wherein the double-stranded deoxyribonucleic acid polynucleotide comprises a protospacer adjacent motif (PAM); and wherein the PAM comprises a sequence according to any one of SEQ ID NOs: 550-567. In some embodiments, the endonuclease further comprises a PI domain comprising a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1277-1641 or 1683, or a variant thereof, or wherein the endonuclease further comprises a PI domain having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to a PI domain of any one of SEQ ID NOs: 1-549 or 602-1276. In some embodiments, the endonuclease further comprises a PI domain comprising a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1, 217, 258, 259, 284, 296, 297, 306, 357, 403, 404, 405, 463, 464, 465, 356, or 484, or a variant thereof. In some embodiments, the endonuclease further comprises a PI domain comprising a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 259, 296, or 484, or a variant thereof. In some embodiments, the endonuclease further comprises a RuvC domain. In some embodiments, the RuvC domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to a RuvC domain of any one of SEQ ID NOs: 1, 217, 258, 259, 284, 296, 297, 306, 357, 403, 404, 405, 463, 464, 465, 356, or 484, or a variant thereof. In some embodiments, the RuvC domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to a RuvC domain of any one of SEQ ID NOs: 259, 296, or 484, or a variant thereof. In some embodiments, the endonuclease further comprises an HNH domain. In some embodiments, the HNH domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to an HNH domain of any one of SEQ ID NOs: 1, 217, 258, 259, 284, 296, 297, 306, 357, 403, 404, 405, 463, 464, 465, 356, or 484, or a variant thereof. In some embodiments, the HNH domain has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to an HNH domain of any one of SEQ ID NOs: 259, 296, or 484, or a variant thereof. In some embodiments, the endonuclease comprises any one of SEQ ID NOs: 1, 217, 258, 259, 284, 296, 297, 306, 357, 403, 404, 405, 463, 464, 465, 356, or 484, or a variant thereof having at least 55%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity thereto. In some embodiments, the engineered guide nucleic acid structure comprises a tracr sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1645-1662 or wherein the engineered guide nucleic acid structure comprises a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to non- degenerate nucleotides of any one of SEQ ID NOs: 568-585 or 1643-1644. In some embodiments, the engineered guide nucleic acid structure comprises a tracr sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1648, 1650, or 1661 or wherein the engineered guide nucleic acid structure comprises a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to non-degenerate nucleotides of any one of SEQ ID NOs: 571, 573, or 584. In some embodiments, the engineered guide nucleic acid structure is configured to form a complex with the endonuclease and comprises (i) a targeting nucleic acid sequence configured to hybridize to a target deoxyribonucleic acid sequence; and (ii) a tracr ribonucleic acid sequence configured to bind to the endonuclease. In some embodiments, the targeting nucleic acid sequence is complementary to a bacterial, archaeal, eukaryotic, fungal, plant, mammalian, or human genomic sequence. In some embodiments, the targeting nucleic acid sequence is 15-24 nucleotides in length.

In some aspects, the present disclosure provides for a method of editing an AAVS1 locus in a cell, comprising contacting to the cell (a) an RNA-guided endonuclease; and (b) an engineered guide nucleic acid structure, wherein the engineered guide nucleic acid structure is configured to form a complex with the endonuclease and the engineered guide nucleic acid structure comprises a spacer sequence configured to hybridize to a region of the AAVS1 locus, wherein the engineered guide nucleic acid structure comprises a targeting sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to at least 18 consecutive nucleotides of any one of SEQ ID NOs: 1665-1666 or a reverse complement thereof. In some embodiments, the engineered guide nucleic acid structure has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NO: 1663 or 1664. In some embodiments, the engineered guide nucleic acid structure is MG71-2-AAVS1-sgRNA-C3 or MG71-2-AAVS1-sgRNA-E2.

In some aspects, the present disclosure provides for a method of editing a TRAC locus in a cell, comprising contacting to the cell (a) an RNA-guided endonuclease; and (b) an engineered guide nucleic acid structure, wherein the engineered guide nucleic acid structure is configured to form a complex with the endonuclease and the engineered guide nucleic acid structure comprises a spacer sequence configured to hybridize to a region of the TRAC locus, wherein the engineered guide nucleic acid structure comprises a targeting sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to at least 18 consecutive nucleotides of any one of SEQ ID NOs: 1668 or 1676-1682, or a reverse complement thereof. In some embodiments, the engineered guide nucleic acid structure has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1667 or 1669-1675. In some embodiments, the engineered guide nucleic acid structure is MG73-1-TRAC-sgRNA-G3, MG89-2-TRAC-sgRNA-F1, MG89-2-TRAC-sgRNA-G5, MG89-2-TRAC-sgRNA-E5, MG89-2-TRAC-sgRNA-F5, MG89-2-TRAC-sgRNA-G1, MG89-2-TRAC-sgRNA-E1, MG89-2-TRAC-sgRNA-B1. In some embodiments, the engineered guide nucleic acid structure is configured to form a complex with the endonuclease and comprises (i) a targeting nucleic acid sequence configured to hybridize to a target deoxyribonucleic acid sequence; and (ii) a tracr ribonucleic acid sequence configured to bind to the endonuclease. In some embodiments, the targeting nucleic acid sequence is 15-24 nucleotides in length. In some embodiments, the engineered guide nucleic acid structure comprises a tracr sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1645-1662 or wherein the engineered guide nucleic acid structure comprises a sequence having at least 80% identity to non-degenerate nucleotides of any one of SEQ ID NOs: 568-585 or 1643-1644. In some embodiments, the engineered guide nucleic acid structure comprises a tracr sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1648, 1650, or 1661 or wherein the engineered guide nucleic acid structure comprises a sequence having at least 80% identity to non-degenerate nucleotides of any one of SEQ ID NOs: 571, 573, or 584. In some embodiments, the RNA guided endonuclease is a class 2, type II Cas endonuclease. In some embodiments, the endonuclease is configured to have selectivity for a PAM sequence comprising any one of SEQ ID NOs: 550-567. In some embodiments, the endonuclease further comprises a PI domain comprising a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1277-1641 or 1683, or a variant thereof, or wherein the endonuclease further comprises a PI domain having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to a PI domain of any one of SEQ ID NOs: 1-549 or 602-1276. In some embodiments, the endonuclease further comprises a PI domain comprising a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1, 217, 258, 259, 284, 296, 297, 306, 357, 403, 404, 405, 463, 464, 465, 356, or 484, or a variant thereof. In some embodiments, the endonuclease further comprises a PI domain comprising a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 259, 296, or 484, or a variant thereof. In some embodiments, the endonuclease comprises any one of SEQ ID NOs: 1, 217, 258, 259, 284, 296, 297, 306, 357, 403, 404, 405, 463, 464, 465, 356, or 484, or a variant thereof having at least 55% identity, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity thereto.

Systems of the present disclosure may be used for various applications, such as, for example, nucleic acid editing (e.g., gene editing), binding to a nucleic acid molecule (e.g., sequence-specific binding). Such systems may be used, for example, for addressing (e.g., removing or replacing) a genetically inherited mutation that may cause a disease in a subject, inactivating a gene in order to ascertain its function in a cell, as a diagnostic tool to detect disease-causing genetic elements (e.g. via cleavage of reverse-transcribed viral RNA or an amplified DNA sequence encoding a disease-causing mutation), as deactivated enzymes in combination with a probe to target and detect a specific nucleotide sequence (e.g. sequence encoding antibiotic resistance int bacteria), to render viruses inactive or incapable of infecting host cells by targeting viral genomes, to add genes or amend metabolic pathways to engineer organisms to produce valuable small molecules, macromolecules, or secondary metabolites, to establish a gene drive element for evolutionary selection, to detect cell perturbations by foreign small molecules and nucleotides as a biosensor.

Lengthy table referenced here
US20240110167A1-20240404-T00001
Please refer to the end of the specification for access instructions.

### EXAMPLES

### Example 1—Metagenomic Analysis for New Proteins

Metagenomic samples were collected from sediment, soil and animal. Deoxyribonucleic acid (DNA) was extracted with a Zymobiomics DNA mini-prep kit and sequenced on an Illumina HiSeq® 2500. Samples were collected with consent of property owners. Additional raw sequence data from public sources included animal microbiomes, sediment, soil, hot springs, hydrothermal vents, marine, peat bogs, permafrost, and sewage sequences. Metagenomic sequence data was searched using Hidden Markov Models generated based on known Cas protein sequences including type II Cas effector proteins. Novel effector proteins identified by the search were aligned to known proteins to identify potential active sites. This metagenomic workflow resulted in delineation of the MG44, MG46, MG71, MG72, MG73, MG74, MG87, MG88 and MG89 families of class II, type II CRISPR endonucleases described herein.

### Example 2 —Discovery of MG44, MG46, MG71, MG72, MG73, MG74, MG87, MG88 and MG89 Families of CRISPR Systems

Analysis of the data from the metagenomic analysis of Example 1 revealed new clusters of previously undescribed putative CRISPR systems comprising 9 families (MG44, MG46, MG71, MG72, MG73, MG74, MG87, MG88 and MG89). The corresponding protein and nucleic acid sequences for these new enzymes and their exemplary subdomains are presented as SEQ ID NOs: 1-549 or 602-1276.

### Example 3 —Determination of Protospacer-Adjacent Motifs

PAM sequences were determined by sequencing plasmids containing randomly-generated PAM sequences that could be cleaved by putative endonucleases expressed in an E. coli lysate-based expression system (myTXTL, Arbor Biosciences). In this system, an E. coli codon optimized nucleotide sequence was transcribed and translated from a PCR fragment under control of a T7 promoter. A second PCR fragment with a tracr sequence under a T7 promoter and a minimal CRISPR array composed of a T7 promoter followed by a repeat-spacer-repeat sequence was transcribed in the same reaction. Successful expression of the endonuclease and tracr sequence in the TXTL system followed by CRISPR array processing provided active in vitro CRISPR nuclease complexes.

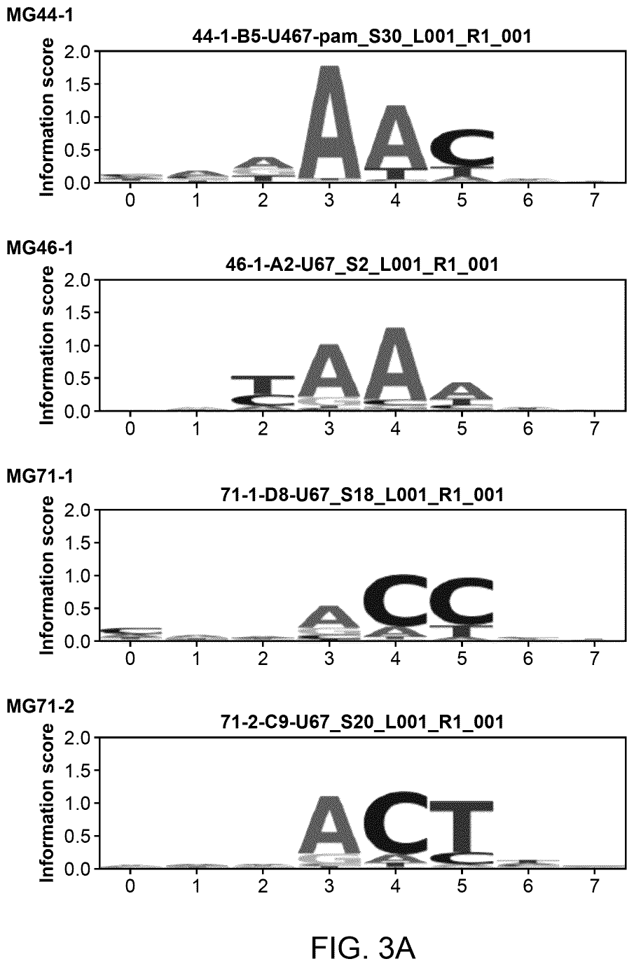

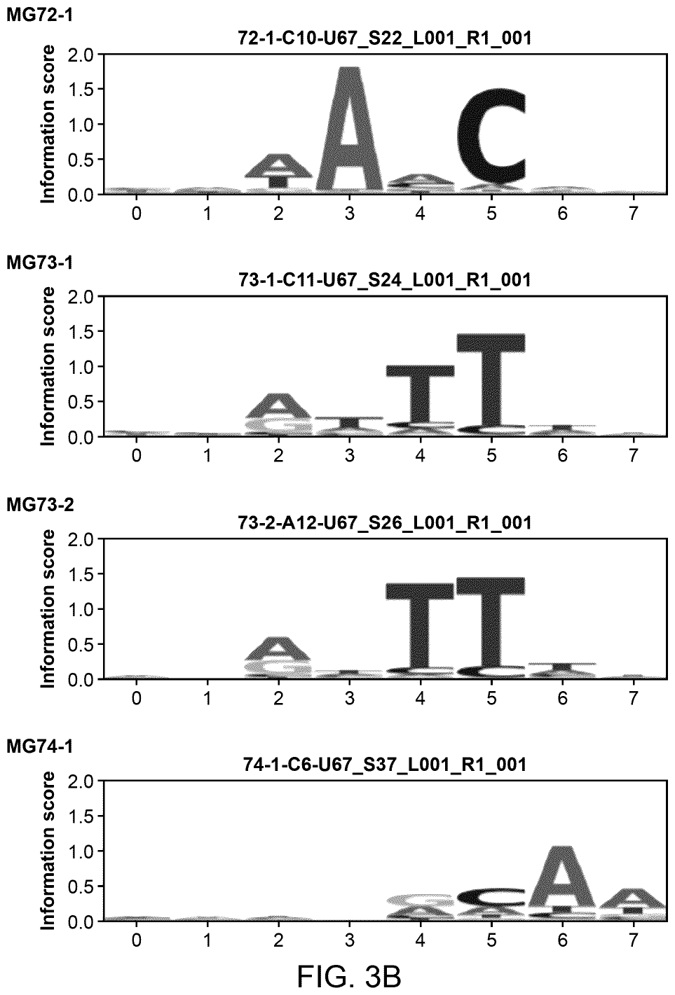

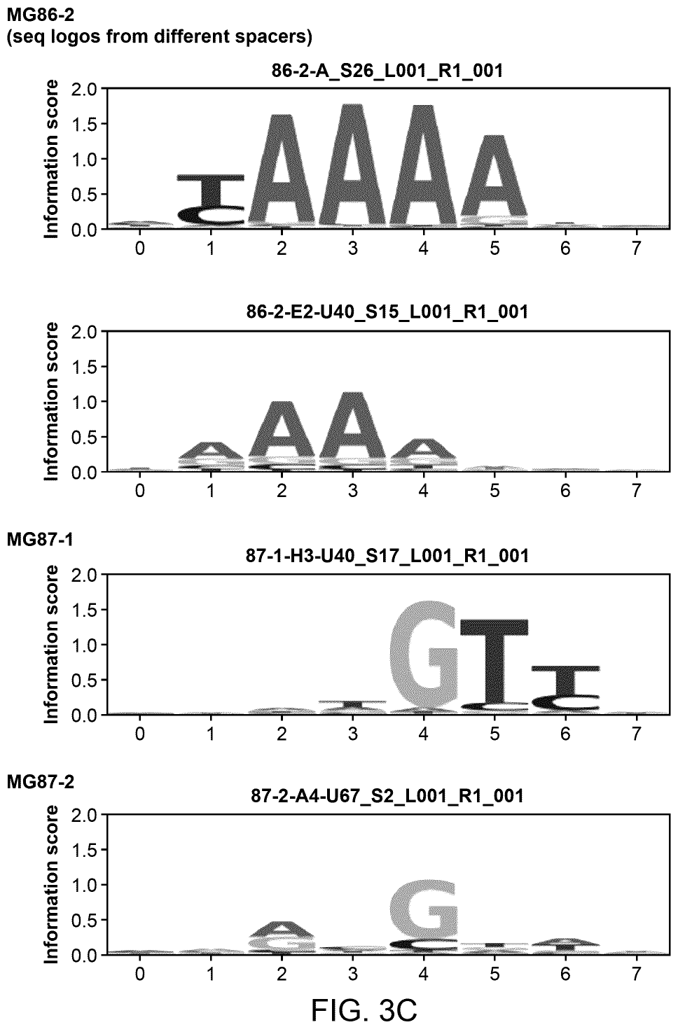

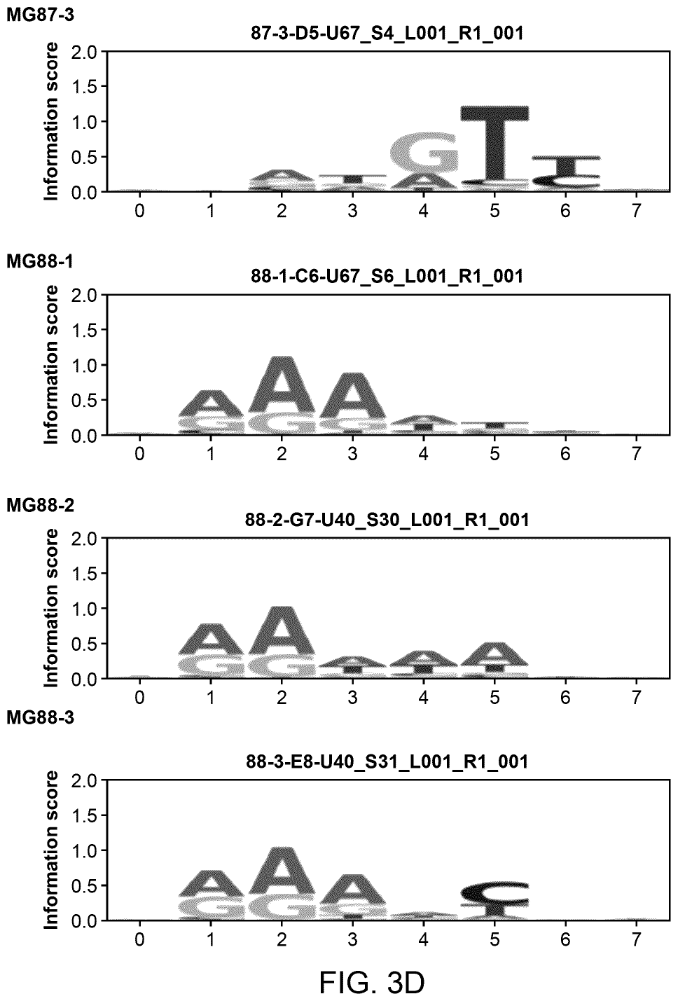

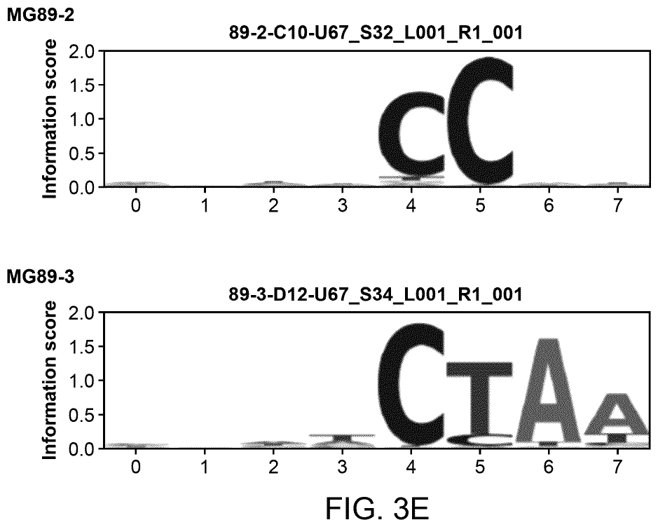

A library of target plasmids containing a spacer sequence matching that in the minimal array followed by 8N mixed bases (putative PAM sequences) was incubated with the output of the TXTL reaction. After 1-3 hr, the reaction was stopped and the DNA was recovered via a DNA clean-up kit, e.g., Zymo DCC, AMPure XP beads, QiaQuick etc. Adapter sequences were blunt-end ligated to DNA with active PAM sequences that had been cleaved by the endonuclease, whereas DNA that had not been cleaved was inaccessible for ligation. DNA segments comprising active PAM sequences were then amplified by PCR with primers specific to the library and the adapter sequence. The PCR amplification products were resolved on a gel to identify amplicons that corresponded to cleavage events. The amplified segments of the cleavage reaction were also used as template for preparation of an NGS library. Sequencing this resulting library, which was a subset of the starting 8N library, revealed the sequences which contain the correct PAM for the active CRISPR complex. For PAM testing with a single RNA construct, the same procedure was repeated except that an in vitro transcribed RNA was added along with the plasmid library and the tracr/minimal CRISPR array template was omitted. For endonucleases where NGS libraries were prepared, seqLogo (see e.g., Huber et al. Nat Methods. 2015 February; 12(2):115-21) representations were constructed and are presented in FIG. 3. The seqLogo module used to construct these representations takes the position weight matrix of a DNA sequence motif (e.g. a PAM sequence) and plots the corresponding sequence logo as introduced by Schneider and Stephens (see e.g. Schneider et al. Nucleic Acids Res. 1990 Oct. 25; 18(20):6097-100. The characters representing the sequence in the seqLogo representations have been stacked on top of each other for each position in the aligned sequences (e.g. PAM sequences). The height of each letter is proportional to its frequency, and the letters have been sorted so the most common one is on top.

### Example 4—(Prophetic) In Vitro Cleavage Efficiency of MG CRISPR Complexes

Endonucleases are expressed as His-tagged fusion proteins from an inducible T7 promoter in a protease deficient E. coli B strain. Cells expressing the His-tagged proteins are lysed by sonication and the His-tagged proteins are purified by Ni-NTA affinity chromatography on a HisTrap FF column (GE Lifescience) on an AKTA Avant FPLC (GE Lifescience). The eluate are resolved by SDS-PAGE on acrylamide gels (Bio-Rad) and are stained with InstantBlue Ultrafast coomassie (Sigma-Aldrich). Purity is determined using densitometry of the protein band with ImageLab software (Bio-Rad). Purified endonucleases are dialyzed into a storage buffer composed of 50 mM Tris-HCl, 300 mM NaCl, 1 mM TCEP, 5% glycerol; pH 7.5 and stored at −80° C.

Target DNAs containing spacer sequences and PAM sequences (determined e.g., as in Example 3) are constructed by DNA synthesis. A single representative PAM is chosen for testing when the PAM has degenerate bases. The target DNAs are comprised 2200 bp of linear DNA derived from a plasmid via PCR amplification with a PAM and spacer located 700 bp from one end. Successful cleavage results in fragments of 700 and 1500 bp. The target DNA, in vitro transcribed single RNA, and purified recombinant protein are combined in cleavage buffer (10 mM Tris, 100 mM NaCl, 10 mM MgCl₂) with an excess of protein and RNA and are incubated for 5 minutes to 3 hours, usually 1 hr. The reaction is stopped via addition of RNAse A and incubation at 60 minutes. The reaction is then resolved on a 1.2% TAE agarose gel and the fraction of cleaved target DNA is quantified in ImageLab software.

### Example 5 —(Prophetic) Testing of Genome Cleavage Activity of MG CRISPR Complexes in E. coli

E. coli lacks the capacity to efficiently repair double-stranded DNA breaks. Thus, cleavage of genomic DNA can be a lethal event. Exploiting this phenomenon, endonuclease activity is tested in E. coli by recombinantly expressing an endonuclease and a tracrRNA in a target strain with spacer/target and PAM sequences which are integrated into its genomic DNA.

In this assay, the PAM sequence is specific for the endonuclease being tested as determined by the methods described in Example 3. sgRNA sequences are determined based upon the sequence and predicted structure of the tracrRNA. Repeat-anti-repeat pairings of 8-12 bp (generally 10 bp) are chosen, starting from the 5′ end of the repeat. The remaining 3′ end of the repeat and 5′ end of the tracrRNA are replaced with a tetraloop. Generally, the tetraloop is GAAA, but other tetraloops can be used, particularly if the GAAA sequence is predicted to interfere with folding. In these cases, a TTCG tetraloop was used.

Engineered strains with PAM sequences integrated into their genomic DNA are transformed with DNA encoding the endonuclease. Transformants are then made chemocompetent and are transformed with 50 ng of single guide RNAs either specific to the target sequence (“on target”), or non-specific to the target (“non target”). After heat shock, transformations are recovered in SOC for 2 hrs at 37° C. Nuclease efficiency is then determined by a 5-fold dilution series grown on induction media. Colonies are quantified from the dilution series in triplicate.

### Example 6 —(Prophetic) Testing of Genome Cleavage Activity of MG CRISPR Complexes in Mammalian Cells

To show targeting and cleavage activity in mammalian cells, the MG Cas effector protein sequences are tested in two mammalian expression vectors: (a) one with a C-terminal SV40 NLS and a 2A-GFP tag, and (b) one with no GFP tag and two SV40 NLS sequences, one on the N-terminus and one on the C-terminus. In some instances, nucleotide sequences encoding the endonucleases are codon-optimized for expression in mammalian cells.

The corresponding single guide RNA sequence (sgRNA) with targeting sequence attached is cloned into a second mammalian expression vector. The two plasmids are cotransfected into HEK293T cells. 72 hr after co-transfection of the expression plasmid and a sgRNA targeting plasmid into HEK293T cells, the DNA is extracted and used for the preparation of an NGS-library. Percent NHEJ is measured via indels in the sequencing of the target site to demonstrate the targeting efficiency of the enzyme in mammalian cells. At least 10 different target sites were chosen to test each protein's activity.

### Example 7 —Gene Editing Outcomes at the DNA Level for TRAC and AAVS1 in K562 Cells

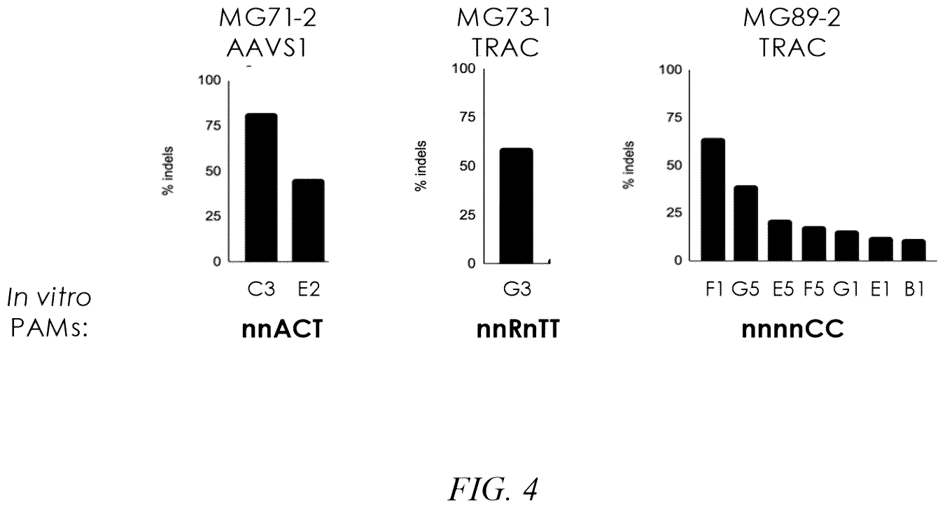

Nucleofection of MG71-2, MG73-1, and MG89-2 mRNA along with the matching guide RNA from Table 4 below (500 ng mRNA/150 pmol guide) was performed into K562 cells (200,000) using the Lonza 4D electroporator. Cells were harvested and genomic DNA prepared three days post-transfection. PCR primers appropriate for use in NGS-based DNA sequencing were generated, optimized, and used to amplify the individual target sequences for each guide RNA. The amplicons were sequenced on an Illumina MiSeq machine and analyzed with a proprietary Python script to measure gene editing (FIG. 4).

TABLE 4
sgRNAs and Associated Sequences Targeted within AAVS1 and TRAC Sites Used
in Example 7

| | SEQ | |
|---|---|---|
| sgRNA | ID NO: | Sequence |
| MG71-2- | 1665 | GGAGAGGGTAGCGCAGGGTG |
| AAVS1- | | |
| target site- | | |
| C3 | | |
| MG71-2- | 1666 | GCCCTGCCAGGAGGGGGCTG |
| AAVS1- | | |
| target site- | | |
| E2 | | |
| MG73-1- | 1668 | TCTTGGTTTTACAGATACGAACCT |
| TRAC- | | |
| target site- | | |
| G3 | | |
| MG89-2- | 1676 | ATATCCAGAACCCTGACCOTGCCG |
| TRAC- | | |
| target site- | | |
| F1 | | |
| MG89-2- | 1677 | GGCCACTTTCAGGAGGAGGATTCG |
| TRAC- | | |
| target site- | | |
| G5 | | |
| MG89-2- | 1678 | CGCAGCGTCATGAGCAGATTAAAC |
| TRAC- | | |
| target site- | | |
| E5 | | |
| MG89-2- | 1679 | CGGCCACTTTCAGGAGGAGGATTC |
| TRAC- | | |
| target site- | | |
| F5 | | |
| MG89-2- | 1680 | GCCGTGTACCAGCTGAGAGACTCT |
| TRAC- | | |
| target site- | | |
| GI | | |
| MG89-2- | 1681 | CCCACAGATATCCAGAACCCTGAC |
| TRAC- | | |
| target site- | | |
| E1 | | |
| MG89-2- | 1682 | ATCCTCTTGTCCCACAGATATCCA |
| TRAC- | | |
| target site- | | |
| B1 | | |
| MG71-2- | 1663 | mG*mG*mA*rGrArGrGrGrUrArGrCrGrCrArGrGrGrUrGrGrUrUrUrGrArGrArGrUrGrArGrArArAr |
| AAVS1- | | UrCrArCrGrArGrUrUrCrArArArArArArCrArUrGrArUrUrUrArUrUrCrArArArCrCrGrUrCrUrUr |
| sgRNA- | | CrUrUrCrGrGrArArGrGrCrCrCrCrArCrArGrUrGrUrGrUrGrGrArCrArGrUrArArArGrCrUrUrGr |
| C3 | | CrUrUrCrGrGrCrArArGrCrU*mU*mU*mU |
| MG71-2- | 1664 | mG*mC*mC*rCrUrGrCrCrArGrGrArCrGrGrGrGrCrUrGrGrUrUrUrGrArGrArGrUrGrArGrArArAr |
| AAVS1- | | UrCrArCrGrArGrUrUrCrArArArArArArCrArUrGrArUrUrUrArUrUrCrArArArCrCrGrUrCrUrUr |
| sgRNA- | | CrUrUrCrGrGrArArGrGrCrCrCrCrArCrArGrUrGrUrGrUrGrGrArCrArGrUrArArArGrCrUrUrGr |
| E2 | | CrUrUrCrGrGrCrArArGrCrUmU*mU*mU |
| MG73-1- | 1667 | mU*mC*mU*rUrGrGrUrUrUrUrArCrArGrArUrArCrGrArArCrCrUrGrUrUrArUrArGrUrGrGrGrAr |
| TRAC- | | ArArUrCrArCrUrArUrArArUrArArGrUrGrArArArUrCrGrCrArArGrGrCrUrCrUrGrUrUrCrUrUr |
| sgRNA- | | GrArArCrArUrCrCrUrUrUrArUrUrArUrArArArArCrUrCrCrUrGrCrCrArArUrCrGrGrUrUrGrGr |
| G3 | | GrArGrU*mU*mU*mU |
| MG89-2- | 1669 | mA*mU*mA*rUrCrCrArGrArArCrCrCrUrGrArCrCrCrUrGrCrCrGrGrUrUrGrUrArGrCrUrUrCrCr |
| TRAC- | | UrUrGrArArGrArArArUrUrCrArArCrGrUrUrGrUrUrArCrArArUrArArGrGrUrUrUrUrCrGrArAr |
| sgRNA- | | ArGrArUrUrArCrCrGrArArCrCrCrGrCrCrCrUrCrArCrUrUrArGrGrUrGrArGrGrGrCrU*mU*mU* |
| F1 | | mU |
| MG89-2- | 1670 | mG*mG*mC*rCrArCrUrUrUrCrArGrGrArGrGrArGrGrArUrUrCrGrGrUrUrGrUrArGrCrUrUrCrCr |
| TRAC- | | UrUrGrArArGrArArArUrUrCrArArCrGrUrUrGrUrUrArCrArArUrArArGrGrUrUrUrUrCrGrArAr |
| sgRNA- | | ArGrArUrUrArCrCrGrArArCrCrCrGrCrCrCrUrCrArCrUrUrArGrGrUrGrArGrGrGrCrU*mU*mU* |
| G5 | | mU |
| MG89-2- | 1671 | mC*mG*mC*rArGrCrGrUrCrArUrGrArGrCrArGrArUrUrArArArCrGrUrUrGrUrArGrCrUrUrCrCr |
| TRAC- | | UrUrGrArArGrArArArUrUrCrArArCrGrUrUrGrUrUrArCrArArUrArArGrGrUrUrUrUrCrGrArAr |
| sgRNA- | | ArGrArUrUrArCrCrGrArArCrCrCrGrCrCrCrUrCrArCrUrUrArGrGrUrGrArGrGrGrCrU*mU*mU* |
| E5 | | mU |
| MG89-2- | 1672 | mC*mG*mG*rCrCrArCrUrUrUrCrArGrGrArGrGrArGrGrArUrUrCrGrUrUrGrUrArGrCrUrUrCrCr |
| TRAC- | | UrUrGrArArGrArArArUrUrCrArArCrGrUrUrGrUrUrArCrArArUrArArGrGrUrUrUrUrCrGrArAr |
| sgRNA- | | ArGrArUrUrArCrCrGrArArCrCrCrGrCrCrCrUrCrArCrUrUrArGrGrUrGrArGrGrGrCrU*mU*mU* |
| F5 | | mU |
| MG89-2- | 1673 | mG*mC*mC*rGrUrGrUrArCrCrArGrCrUrGrArGrArGrArCrUrCrUrGrUrUrGrUrArGrCrUrUrCrCr |
| TRAC- | | UrUrGrArArGrArArArUrUrCrArArCrGrUrUrGrUrUrArCrArArUrArArGrGrUrUrUrUrCrGrArAr |
| sgRNA- | | ArGrArUrUrArCrCrGrArArCrCrCrGrCrCrCrUrCrArCrUrUrArGrGrUrGrArGrGrGrCrU*mU*mU* |
| G1 | | mU |
| MG89-2- | 1674 | mC*mC*mC*rArCrArGrArUrArUrCrCrArGrArArCrCrCrUrGrArCrGrUrUrGrUrArGrCrUrUrCrCr |
| TRAC- | | UrUrGrArArGrArArArUrUrCrArArCrGrUrUrGrUrUrArCrArArUrArArGrGrUrUrUrUrCrGrArAr |
| sgRNA- | | ArGrArUrUrArCrCrGrArArCrCrCrGrCrCrCrUrCrArCrUrUrArGrGrUrGrArGrGrGrCrU*mU*mU* |
| E1 | | mU |
| MG89-2- | 1675 | mA*mU*mC*rCrUrCrUrUrGrUrCrCrCrArCrArGrArUrArUrCrCrArGrUrUrGrUrArGrCrUrUrCrCr |
| TRAC- | | UrUrGrArArGrArArArUrUrCrArArCrGrUrUrGrUrUrArCrArArUrArArGrGrUrUrUrUrCrGrArAr |
| sgRNA- | | ArGrArUrUrArCrCrGrArArCrCrCrGrCrCrCrUrCrArCrUrUrArGrGrUrGrArGrGrGrCrU*mU*mU* |
| B1 | | mU |

(r= native ribose base, m= 2'-O methyl modified base, F= 2' Fluro modified base, *= phosphorothioate bond)

Systems of the present disclosure may be used for various applications, such as, for example, nucleic acid editing (e.g., gene editing), binding to a nucleic acid molecule (e.g., sequence-specific binding). Such systems may be used, for example, for addressing (e.g., removing or replacing) a genetically inherited mutation that may cause a disease in a subject, inactivating a gene in order to ascertain its function in a cell, as a diagnostic tool to detect disease-causing genetic elements (e.g. via cleavage of reverse-transcribed viral RNA or an amplified DNA sequence encoding a disease-causing mutation), as deactivated enzymes in combination with a probe to target and detect a specific nucleotide sequence (e.g. sequence encoding antibiotic resistance int bacteria), to render viruses inactive or incapable of infecting host cells by targeting viral genomes, to add genes or amend metabolic pathways to engineer organisms to produce valuable small molecules, macromolecules, or secondary metabolites, to establish a gene drive element for evolutionary selection, to detect cell perturbations by foreign small molecules and nucleotides as a biosensor.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

LENGTHY TABLES
The patent application contains a lengthy table section. A copy of the table is available in electronic form from the USPTO web site (https://seqdata.uspto.gov/?pageRequest=docDetail&DocID=US20240110167A1). An electronic copy of the table will also be available from the USPTO upon request and payment of the fee set forth in 37 CFR 1.19(b)(3).

1-105. (canceled)

106. An engineered nuclease system comprising:
(a) an endonuclease configured to have selectivity for a protospacer adjacent motif (PAM) sequence comprising nnnnCC, wherein said endonuclease is a class 2, type II Cas endonuclease and said endonuclease comprises a PAM-Interacting (PI) domain having at least 80% amino acid sequence identity to SEQ ID NO: 1633; and
(b) an engineered guide nucleic acid structure configured to form a complex with said endonuclease, wherein said engineered guide nucleic acid structure comprises:
(i) a targeting nucleic acid sequence configured to hybridize to a target deoxyribonucleic acid sequence; and
(ii) a tracr ribonucleic acid sequence configured to bind to said endonuclease.

107. The engineered nuclease system of claim 106, wherein said endonuclease further comprises one or more of: a RuvC domain or an HNH domain.

108. The engineered nuclease system of claim 107, wherein said RuvC domain comprises an amino acid sequence having at least 80% sequence identity to a RuvC domain of SEQ ID NO: 484.

109. The engineered nuclease system of claim 107, wherein said HNH domain comprises an amino acid sequence having at least 80% sequence identity to an HNH domain of SEQ ID NO: 484.

110. The engineered nuclease system of claim 106, wherein said tracr ribonucleic acid sequence comprises a sequence having at least 80% sequence identity to SEQ ID NO: 1661.

111. The engineered nuclease system of claim 106, wherein said engineered guide nucleic acid structure comprises a sequence having at least 80% sequence identity to SEQ ID NO: 584.

112. The engineered nuclease system of claim 106, wherein said targeting nucleic acid sequence comprises a spacer sequence configured to hybridize to a region of a TRAC locus or a region of an AAVS1 locus.

113. The engineered nuclease system of claim 112, wherein said engineered guide nucleic acid structure comprises a sequence having at least 80% sequence identity to any one of SEQ ID NOs: 1669-1675.

114. The engineered nuclease system of claim 112, wherein said target deoxyribonucleic acid sequence comprises a sequence having at least 80% sequence identity to at least 18 consecutive nucleotides of any one of SEQ ID NOs: 1676-1682, or a reverse complement thereof.

115. The engineered nuclease system of claim 106, wherein said endonuclease comprises an amino acid sequence comprising at least 80% sequence identity to SEQ ID NO: 484.

116. A method of editing a locus in a cell, said method comprising contacting to said cell:
(a) an endonuclease or a polynucleotide encoding said endonuclease, wherein said endonuclease is a class 2, type II Cas endonuclease, wherein said endonuclease is configured to have selectivity for a protospacer adjacent motif (PAM) sequence comprising nnnnCC and comprises a PAM-Interacting (PI) domain comprising an amino acid sequence having at least 80% sequence identity to SEQ ID NO: 1633; and
(b) an engineered guide nucleic acid structure or one or more polynucleotides encoding said engineered guide nucleic acid structure, wherein said engineered guide nucleic acid structure is configured to form a complex with said endonuclease and comprises:
(i) a targeting nucleic acid sequence configured to hybridize to a target deoxyribonucleic acid sequence in said cell; and
(ii) a tracr ribonucleic acid sequence configured to bind to said endonuclease.

117. The method of claim 116, wherein said endonuclease further comprises one or more of: a RuvC domain or an HNH domain.

118. The method of claim 117, wherein said RuvC domain comprises an amino acid sequence having at least 80% sequence identity to a RuvC domain of SEQ ID NO: 484.

119. The method of claim 117, wherein said HNH domain comprises an amino acid sequence having at least 80% sequence identity to an HNH domain of SEQ ID NO: 484.

120. The method of claim 116, wherein said tracr ribonucleic acid sequence comprises a sequence having at least 80% sequence identity to SEQ ID NO: 1661.

121. The method of claim 116, wherein said engineered guide nucleic acid structure comprises a sequence having at least 80% sequence identity to SEQ ID NO: 584.

122. The method of claim 116, wherein said locus is a TRAC locus or an AAVS1 locus.

123. The method of claim 122, said engineered guide nucleic acid structure comprises a sequence having at least 80% sequence identity to any one of SEQ ID NOs: 1669-1675.

124. The method of claim 122, wherein said target deoxyribonucleic acid sequence comprises a sequence having at least 80% sequence identity to at least 18 consecutive nucleotides of any one of SEQ ID NOs: 1676-1682, or a reverse complement thereof.

125. The method of claim 116, wherein said endonuclease comprises an amino acid sequence comprising at least 80% sequence identity to SEQ ID NO: 484.
